(19) United States
(12) Patent Application Publication
(10) Pub. No.: US 2026/0240855 A1
(43) Pub. Date: Aug. 20, 2026

(54) DOSAGE REGIMEN FOR SOTORASIB/CARBOPLATIN/PEMETREXED IN CANCER TREATMENT

(71) Applicant: AMGEN INC., Thousand Oaks, CA (US)

(72) Inventors: Bhakti MEHTA, Thousand Oaks, CA (US); Haby HENARY, Moorpark, CA (US); Gataree NGARMCHAMNANRITH, Los Angeles, CA (US); Igor Ivanovich RYBKIN, Thousand Oaks, CA (US); Joseph PARK, Newbury Park, CA (US)

(73) Assignee: AMGEN INC., Thousand Oaks, CA (US)

(21) Appl. No.: 19/472,476

(22) PCT Filed: Apr. 12, 2024
(86) PCT No.: PCT/US2024/024485

§ 371 Date: Oct. 3, 2025

Related U.S. Application Data
(60) Provisional application No. 63/459,349, filed on Apr. 14, 2023.
(60) Provisional application No. 63/520,050, filed on Aug. 16, 2023.

(51) Int. Cl. A61K 31/519; A61K 31/555; A61P 35/00

(52) U.S. Cl. CPC A61K 31/519; A61K 31/555; A61P 35/00

(57) ABSTRACT

Provided herein are methods of treating cancer comprising a KRAS G12C mutation in a subject in need thereof comprising administering to the subject sotorasib, carboplatin and pemetrexed in amounts effective to treat the cancer.

### CROSS-REFERENCE TO RELATED APPLICATIONS

This application claims the benefit of priority to U.S. Provisional Patent Application No. 63/459,349, filed Apr. 18, 2023, and U.S. Provisional Patent Application No. 63/520,050, filed Aug. 16, 2023, which is hereby incorporated by reference in its entirety.

### BACKGROUND

The rat sarcoma (RAS) proto-oncogene has been identified as an oncogenic driver of tumorigenesis in cancers, such as non-small cell lung cancer (NSCLC) and colorectal cancer (CRC). The RAS family consists of 3 closely related genes that express guanosine triphosphate (GTP)-ases responsible for regulating cellular proliferation and survival. The RAS proteins, Kirsten rat sarcoma viral oncogene homolog (KRAS), Harvey rat sarcoma viral oncogene homolog (HRAS), and neuroblastoma RAS viral oncogene homolog (NRAS) can be mutationally activated at codons 12, 13, or 61, leading to human cancers. Different tumor types are associated with mutations in certain isoforms of RAS, with KRAS being the most frequently mutated isoform in most cancers. While the role of KRAS mutations in human cancers has been known for decades, no anti-cancer therapies specifically targeting KRAS mutations have been successfully developed, until recently, largely because the protein had been considered intractable for inhibition by small molecules.

As further detailed in this disclosure, sotorasib is a small molecule that irreversibly inhibits the KRAS^{G12C} protein. In 2021, LUMAKRAS was approved by the US Food and Drug Administration (FDA) for the treatment of certain KRAS G12C mutated lung cancers, making it the first compound in its class approved by a regulatory agency anywhere in the world. While sotorasib monotherapy appears to drive substantial anti-tumor activity, combination therapies are desirable to improve treatment options for patients.

### SUMMARY

Described herein are methods of treating cancer comprising a KRAS G12C mutation in a subject in need thereof comprising administering to the subject (a) a first regimen comprising (i) a therapeutically effective amount of sotorasib, (ii) a therapeutically effective amount of carboplatin and (iii) a therapeutically effective amount of pemetrexed and then (b) a second regimen comprising (i) the therapeutically effective amount of sotorasib and (ii) the therapeutically effective amount of pemetrexed. In various embodiments, the therapeutically effective amount of sotorasib is 240 mg. In various embodiments, the therapeutically effective amount of sotorasib is 960 mg. In some various embodiments, the subject, prior to treatment, has not received a prior systemic anticancer therapy for the cancer (first line treatment). In various embodiments, the cancer exhibits a tumor cell (TC) score of less than 1% or a tumor proportion score (TPS) of less than 1%.

### DETAILED DESCRIPTION

Provided herein are methods of treating cancer comprising a KRAS G12C mutation in a subject in need thereof comprising administering to the subject (a) a first regimen comprising (i) a therapeutically effective amount of sotorasib, (ii) a therapeutically effective amount of carboplatin and (iii) a therapeutically effective amount of pemetrexed and then (b) a second regimen comprising (i) the therapeutically effective amount of sotorasib and (ii) the therapeutically effective amount of pemetrexed. In various embodiments, the therapeutically effective amount of sotorasib is 240 mg. In various embodiments, the therapeutically effective amount of sotorasib is 960 mg.

In various embodiments, the first regimen is administered for a period of 1 to 10 cycles (e.g., 1, 2, 3, 4, 5, 6, 7, 8, 9, or 10 cycles). In various embodiments, the first regimen is administered for a period of 4 cycles. In various embodiments, the cycle is a period of 14 to 28 days or 18 to 24 days. In some embodiments, the cycle is a period of 14, 15, 16, 17, 18, 19, 20, 21, 22, 23, 24, 25, 26, 27 or 28 days. In various embodiments, the cycle is a period of 21 days.

Provided herein are methods of treatment regarding the administration of two or more therapeutics (e.g., sotorasib, carboplatin and pemetrexed). Unless otherwise described herein, combination therapy of two or more therapeutics as discussed herein include both concomitant and sequential administration.

### Definitions

The term “adult” as used herein refers to a subject of 18 or more years, or legal adult age within the country the subject is located when treated with one or methods disclosed herein, whichever is older.

The term “pharmaceutically acceptable” as used herein refers to generally recognized for use in subjects, particularly in humans.

The term “pharmaceutically acceptable salt” as used herein refers to a salt of a compound that is pharmaceutically acceptable and that possesses the desired pharmacological activity of the parent compound.

Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, and the like. Additional examples of such salts can be found in Berge et al., J. Pharm. Sci. 66(1):1-19 (1977). See a/so Stahl et al., Pharmaceutical Salts: Properties, Selection, and Use, 2nd Revised Edition (2011).

The term “prior systemic anticancer therapy” as used herein refers to a systemic anticancer therapy (including but not limited to chemotherapy, antibody therapy, molecular targeted therapy, hormonal therapy, retinoid therapy, or any investigational anticancer agent) within 2 years of the start of treatment with any of the methods disclosed herein, except for (i) prior or ongoing anticancer hormonal therapy given in adjuvant settings after completely resected early-stage breast cancer with no known active disease for over 2 years or (ii) prior neoadjuvant/adjuvant therapy (to the curative surgery) systemic anticancer therapy for early-stage non-small cell lung cancer (NSCLC), with or without anti-PD-1/L1 therapy, which was completed greater than 6 month before the diagnosis of the cancer intended for being treated with one or more methods disclosed herein.

In some embodiments, the prior systemic anticancer therapy is a therapy with a KRAS^{G12C} inhibitor. In some embodiments, KRAS^{G12C} inhibitor is sotorasib, adagrasib, GDC-6036, D-1553, JDQ443, LY3484356, BI1823911, JAB-21822, RMC-6291, or APG-1842. In some embodiments, the KRAS^{G12C} inhibitor is sotorasib. In some embodiments, the KRAS^{G12C} inhibitor is adagrasib.

The term “subject” or “subjects” are used interchangeably with “patient” or “patients.”

### Sotorasib

Provided herein are methods of treating cancer comprising a KRAS G12C mutation in a subject in need thereof comprising administering to the subject (a) a first regimen comprising (i) a therapeutically effective amount of sotorasib, (ii) a therapeutically effective amount of carboplatin and (iii) a therapeutically effective amount of pemetrexed and then (b) a second regimen comprising (i) the therapeutically effective amount of sotorasib and (ii) the therapeutically effective amount of pemetrexed. In various embodiments, the therapeutically effective amount of sotorasib is 240 mg. In some embodiments, sotorasib is administered before carboplatin, when administered on the same day, i.e., day 1 of each cycle of the first regimen.

Sotorasib is a small molecule that irreversibly inhibits the KRAS^{G12C} protein. Sotorasib is also referred to as AMG 510 or 6-fluoro-7-(2-fluoro-6-hydroxyphenyl)-(1M)-1-[4-methyl-2-(propan-2-yl)pyridin-3-yl]-4-[(2S)-2-methyl-4-(prop-2-enoyl)piperazin-1-yl]pyrido[2,3-d]pyrimidin-2(1H)-one and sold as a pharmaceutical composition in tablet form under the name LUMAKRAS® (e.g., in the United States) or LUMYKRAS® (e.g., in Europe). Sotorasib has the following structure:

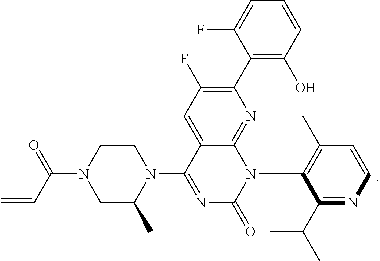

Sotorasib binds to the P2 pocket of KRAS adjacent to the mutant cysteine at position 12 and the nucleotide-binding pocket. The inhibitor contains a thiol reactive portion which covalently modifies the cysteine residue and locks KRAS^{G12C} in the inactive guanosine diphosphate (GDP)-bound conformation (Canon et al., 2019). This blocks the interaction of KRAS with effectors like rapidly accelerated fibrosarcoma (RAF), thus preventing downstream signaling, including the phosphorylation of extracellular signal-regulated kinase (ERK) (Simanshu et al., 2017; Ostrem et al., 2013). Inactivation of KRAS^{G12C} through small molecule inhibitors has previously demonstrated suppression of cell growth and induction of apoptosis in tumor cell lines and xenografts with the KRAS G12C mutation (Janes et al., 2018; Ostrem and Shokat, 2016; Patricelli et al., 2016). Studies of sotorasib have demonstrated inhibition of growth and regression of cells and tumors harboring KRAS^{G12C} (Canon et al., 2019). See also, LUMAKRAS® US Prescribing Information, Amgen Inc., Thousand Oaks, California, 91320 (revision 01/2023), which is herein incorporated by reference in its entirety.

Provided herein are methods comprising administering sotorasib as a free base. In various embodiments, sotorasib is administered as a pharmaceutically acceptable salt. For clarity the term “sotorasib” as used herein refers to the free base of sotorasib. Any method described herein referencing sotorasib can also be practiced using a pharmaceutically acceptable salt of sotorasib. In some embodiments, sotorasib can be administered as a hydrochloride, phosphate, or mesylate. In some embodiments, sotorasib can be administered as a hydrochloride. In some embodiments, sotorasib can be administered as a phosphate. In some embodiments, sotorasib can be administered as a mesylate. For clarity, if a method provided herein recites, e.g., the administration of 240 mg (or 960 mg) of sotorasib or a pharmaceutically acceptable salt thereof to a subject, the method calls for the administration 240 mg (or 960 mg) of a free base of sotorasib or the amount of a pharmaceutically acceptable salt that corresponds to the administration of 240 mg (or 960 mg) of free base of sotorasib.

Further provided herein are methods, wherein sotorasib is administered orally. In various embodiments, sotorasib is administered once daily. In some embodiments, the total daily dose of sotorasib (e.g., 240 mg) is administered in two equal doses twice daily (e.g., 2×120 mg). In various embodiments, sotorasib is administered as a solid dosage form. In some embodiments, the solid dosage form is a tablet. In various embodiments, 240 mg of sotorasib is administered as one tablet comprising 240 mg of sotorasib or two tablets each comprising 120 mg of sotorasib. In various embodiments, 960 mg of sotorasib is administered as three tablets each comprising 320 mg of sotorasib, four tablets each comprising 240 mg of sotorasib, or eight tablets each comprising 120 mg of sotorasib.

In various embodiments, sotorasib is taken at approximately the same time each day with or without food. In some embodiments, sotorasib tablets are dispersed in 120 mL (4 ounces) of non-carbonated, room-temperature water in a container without crushing. The liquid is stirred for approximately 3 minutes until the tablets are dispersed into small pieces and administered to the subject immediately or within 2 hours. Afterwards, the container is rinsed with an additional 120 mL (4 ounces) of water and administered to the subject. This method may be used to administer sotorasib to subjects who have difficulty swallowing solid dosage forms, e.g., tablets.

### Carboplatin

Provided herein are methods of treating cancer comprising a KRAS G12C mutation in a subject in need thereof comprising administering to the subject (a) a first regimen comprising (i) a therapeutically effective amount of sotorasib, (ii) a therapeutically effective amount of carboplatin and (iii) a therapeutically effective amount of pemetrexed and then (b) a second regimen comprising (i) the therapeutically effective amount of sotorasib and (ii) the therapeutically effective amount of pemetrexed. In various embodiments, the therapeutically effective amount of sotorasib is 240 mg. In various embodiments, the therapeutically effective amount of sotorasib is 960 mg.

Carboplatin is a platinum coordination compound. The chemical name for carboplatin is platinum, diammine[1,1-cyclobutane-dicarboxylato(2-)-0,0′]-, (SP-4-2) or cis-(1,1-cyclobutanedicarboxylato)-diamine-platinum(II), and has the following structural formula:

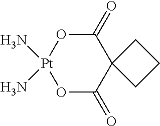

Carboplatin acts by crosslinking deoxyribonucleic acid (DNA) strands, thereby inhibiting DNA synthesis and function. Carboplatin was introduced in 1981 as an analogue of cisplatin, which possessed reduced nonhematological toxicities when compared with cisplatin in experimental models. Calvert et al., 1989. In certain cases, platinum-containing chemotherapy has been the front-line regimen for subjects with advanced or metastatic non-small cell lung cancer (NSCLC) without genomic epithelial growth factor receptor (EGFR) or anaplastic lymphoma kinase (ALK) tumor aberrations until the recent introduction of immunotherapy in combination with chemotherapy (e.g., pembrolizumab in combination with pemetrexed and platinum chemotherapy; see KEYTRUDA® US Prescribing Information, Merck & Co., Kenilworth, NJ 07033 (revision 3/2023), which is herein incorporated by reference in its entirety). The other chemotherapy agent combined with platinum may include pemetrexed (for nonsquamous NSCLC), gemcitabine (for squamous NSCLC), or taxanes (paclitaxel, nab-paclitaxel, or docetaxel).

### Carboplatin Dosing

Carboplatin is commercially available as PARAPLATIN®, BLASTOCARB®, BLASTOPLATIN®, CARBOKEM®, CARBOMAX®, PARAPLATIN®, CARBOPA®, KARPLAT®, and others. Complete information about carboplatin preparation, dispensing, dosage, and administration schedule can be found in the local package insert (for the United States, see, e.g., CARBOplatin Injection, U.S. Prescribing Information, Fresenius KABI, Lake Zurich, Illinois, 60047 (revision 5/2021), which is herein incorporated by reference in its entirety).

Provided herein are methods comprising the administration of a therapeutically effective amount of carboplatin. The Calvert formula [total dose of carboplatin (mg)=(target area under the concentration-time curve (AUC) X (glomerular filtration rate (GFR)+25)] is used for the calculation of the therapeutically effective amount of carboplatin. In some embodiments, the therapeutically effective amount of carboplatin is an amount corresponding to 5 mg/(mL×min) (AUC 5) multiplied with the subject's GFR in mL/min increased by 25 mL/min. The GFR used in the Calvert formula to calculate AUC-based dosing should not exceed 125 mL/min, calculated using the Cockcroft-Gault formula (see Calvert et al., 1989; Cockcroft et al., 1976; and Example 2 below). For example, based on a target carboplatin AUC of 5, the maximum dose of carboplatin is 750 mg (i.e., 5 mg/mL/min×150 mL/min). Accordingly, provided herein are also methods, wherein the therapeutically effective amount of carboplatin is 750 mg or less.

In various embodiments, carboplatin is administered intravenously.

Carboplatin is administered during the first regimen, but not during the second regimen of the methods provided herein, i.e., provided herein are methods, wherein the subject is not administered carboplatin during the second regimen. In various embodiments, the first regimen is administered for a number of cycles. In some embodiments, the first regimen is administered for 4 cycles (21 days each). In various embodiments, carboplatin is administered on day 1 of each cycle during the first regimen. In some embodiments, sotorasib is administered before carboplatin, when administered on the same day, i.e., day 1 of each cycle of the first regimen.

### Pemetrexed and Other Medications

Provided herein are methods of treating cancer comprising a KRAS G12C mutation in a subject in need thereof comprising administering to the subject (a) a first regimen comprising (i) a therapeutically effective amount of sotorasib, (ii) a therapeutically effective amount of carboplatin and (iii) a therapeutically effective amount of pemetrexed and then (b) a second regimen comprising (i) the therapeutically effective amount of sotorasib and (ii) the therapeutically effective amount of pemetrexed. In various embodiments, the therapeutically effective amount of sotorasib is 240 mg. In various embodiments, the therapeutically effective amount of sotorasib is 960 mg.

Also provided herein are methods further comprising other medications to the subject. In various embodiments, the methods provided herein are methods further administering to the subject a therapeutically effective amount of folic acid. In various embodiments, the methods provided herein are methods, further comprising administering to the subject a therapeutically effective amount of vitamin B12. In various embodiments, the methods provided herein are methods, further comprising administering to the subject a therapeutically effective amount of a corticosteroid, such as dexamethasone or an equivalent thereof.

Pemetrexed is a folate analog metabolic inhibitor that disrupts folate-dependent metabolic processes essential for cell replication. Pemetrexed is approved by the FDA, e.g., in combination with pembrolizumab and platinum chemotherapy (carboplatin or cisplatin), as initial treatment of subjects with metastatic nonsquamous NSCLC with no EGFR or ALK tumor genomic aberrations.

Pemetrexed is commercially available as ALIMTA®, and others. Complete information about pemetrexed preparation, dispensing, dosage, and administration schedule can be found in the local package insert (for the United States, see, e.g., ALIMTA® U.S. Prescribing Information, Lilly USA, LLC, Indianapolis, Indiana 46285 (revision 8/2022), which is herein incorporated by reference in its entirety). The drug substance of ALIMTA (pemetrexed for injection) is pemetrexed disodium heptahydrate, has the chemical name L-glutamic acid, N-[4-[2-(2-amino-4,7-dihydro-4-oxo-1H-pyrrolo[2,3-d]pyrimidin-5-yl)ethyl]benzoyl]-, disodium salt, heptahydrate with a molecular formula of C₂₀H₁₉N₅Na₂O₆·7H₂O and a molecular weight of 597.49. The structural formula is as follows:

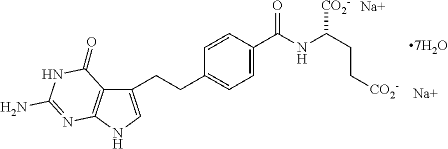

ALIMTA is a sterile white-to-light yellow or green-yellow lyophilized powder in single-dose vials to be reconstituted for intravenous infusion.

### Pemetrexed Dosing

Provided herein are methods, wherein the therapeutically effective amount of pemetrexed is 500 mg/m². In various embodiments, pemetrexed is administered intravenously. In some embodiments, pemetrexed is administered intravenously over 10 minutes.

In the methods provided herein, pemetrexed is administered on day 1 of the first cycle of the first regimen and then in 21 day intervals (i.e., on Day 1, Day 22, Day 43, etc.) throughout the course of treatment, i.e., until the administration of pemetrexed ends. In various embodiments, pemetrexed and carboplatin are administered both on day 1 of each cycle during the administration of the first regimen. In some embodiments, after the administration of carboplatin has ceased, pemetrexed is administered in 21 day intervals. In some embodiments, sotorasib is administered before pemetrexed, when administered on the same day, e.g., day 1 of each cycle of the first regimen and day 1 of the second regimen.

In various embodiments, pemetrexed is administered to subjects with a creatinine clearance (CrCl, calculated by the Cockcroft-Gault equation (see Cockcroft et al., 1976; and Example 2 below)) of 45 mL/min or greater.

Accordingly, provided herein are methods, wherein the administration of pemetrexed starts on day 1 of the first regimen. Further provided herein are methods, wherein the pemetrexed is administered in 21 day intervals during the period the first regimen and the second regimen is administered to the subject.

### Other Medications and Their Dosing

As discussed above, also provided herein are methods, further comprising administering to the subject a therapeutically effective amount of folic acid; and methods, further comprising administering to the subject a therapeutically effective amount of vitamin B₁₂. See, e.g., ALIMTA® U.S. Prescribing Information, Lilly USA, LLC, Indianapolis, Indiana 46285 (revision 8/2022)).

Provided herein are methods, wherein the therapeutically effective amount of folic acid is 350 μg to 1000 μg once daily. In some embodiments, the therapeutically effective amount of folic acid is 400 μg once daily. In various embodiments, the therapeutically effective amount of folic acid is 400 μg to 1000 μg once daily. In various embodiments, the folic acid administration commences 7 days prior to pemetrexed administration and ends 21 days after cessation of pemetrexed administration.

Also provided herein are methods, wherein the therapeutically effective amount of vitamin B₁₂ is 1 mg. In various embodiments, the vitamin B12 is administered in the week prior to first pemetrexed administration and once every 9 weeks (±2 weeks) thereafter until cessation of pemetrexed administration. In some embodiments, the vitamin B₁₂ is administered one week prior to first pemetrexed administration and once every 9 weeks thereafter until cessation of pemetrexed administration. In various embodiments, the vitamin B12 is administered intramuscularly.

As discussed above, also provided herein are methods, further comprising administering to the subject a therapeutically effective amount of a corticosteroid, such as dexamethasone or an equivalent thereof.

Skin rash due to pemetrexed has been reported more frequently in subjects not pretreated with a corticosteroid. Pretreatment with corticosteroids (dexamethasone or equivalent) reduces the incidence and severity of cutaneous reaction. In clinical studies, dexamethasone 4 mg was given by mouth twice daily the day before, the day of, and the day after pemetrexed administration. See, e.g., ALIMTA® U.S. Prescribing Information, Lilly USA, LLC, Indianapolis, Indiana 46285 (as approved Aug. 19, 2004), available at https://www.accessdata.fda.gov/drugsatfda_docs/label/2004/021677lbl.pdf, last accessed Mar. 30, 2023). Accordingly, also provided herein are methods further comprising administering to the subject a therapeutically effective amount of a corticosteroid. In some embodiments, the corticosteroid is dexamethasone or an equivalent thereof. In some embodiments, the corticosteroid is dexamethasone. In various embodiments, the therapeutically effective amount of dexamethasone is 4 mg twice daily. In some embodiments, the dexamethasone is administered the day before, the day of, and the day after each pemetrexed administration to the subject.

### Concomitant Therapies

In various embodiments, the subject is in further need of treatment with an acid-reducing agent. Acid-reducing agents include, but are not limited to, a proton pump inhibitor (PPI), a H2 receptor antagonist (H2RA), and a locally acting antacid. In some embodiments, the subject is further in need of treatment with a PPI or a H2RA. Exemplary PPIs include, but are not limited to, omeprazole, pantoprazole, esomeprazole, lansoprazole, rabeprazole, or dexlansoprazole. Exemplary H2RAs include, but are not limited to, famotidine, ranitidine, cimetidine, nizatidine, roxatidine and lafutidine. Exemplary locally acting antacids include, but are not limited to, sodium bicarbonate, calcium carbonate, aluminum hydroxide, and magnesium hydroxide. In some embodiments, the subject, who is in further need of treatment with an acid-reducing agent, is not administered a proton pump inhibitor or a H2 receptor antagonist in combination with sotorasib. In some embodiments, the subject, who is in further need of treatment with an acid-reducing agent, is not administered a proton pump inhibitor or a H2 receptor antagonist in combination with sotorasib, but is administered a locally acting antacid in combination with sotorasib. In some embodiments, sotorasib is administered about 4 hours before or about 10 hours after a locally acting antacid. In some embodiments, if the subject is in further need of treatment with an acid-reducing agent, the subject is administered sotorasib with an acidic beverage (such as cola). In some embodiments, the subject has not been administered a PPI or H2RA within 7 days before the first regimen starts, i.e., before cycle 1 day 1 of the first regimen.

In various embodiments, the subject is in further need of treatment with a CYP3A4 inducer. In some embodiments, the subject is not administered a CYP3A4 inducer in combination with sotorasib. Exemplary CYP3A4 inducers include, but are not limited to, barbiturates, brigatinib, carbamazepine, clobazam, dabrafenib, efavirenz, elagolix, enzalutamide, eslicarbazepine, glucocorticoids, letermovir, lorlatinib, modafinil, nevirapine, oritavancin, oxcarbazepine, perampanel, phenobarbital, phenytoin, pioglitazone, rifabutin, rifampin, telotristat, and troglitazone. See, e.g., Flockhart DA, Drug Interactions: Cytochrome P450 Drug Interaction Table. Indiana University School of Medicine (2007), www.drug-interactions.medicine.iu.edu, accessed May 2021. In some embodiments, the subject is not administered a strong CYP3A4 inducer in combination with sotorasib. Exemplary strong CYP3A4 inducers include, but are not limited to, phenytoin and rifampin. See, e.g., www.fda.gov/drugs/drug-interactions-labeling/drug-development-and-drug-interactions-table-substrates-inhibitors-and-inducers, accessed May 2021. In some embodiments, strong CYP3A4 inducers include, but are not limited to, rifampin, phenytoin, mitotane, carbamazepine, avasimibe, enzalutamide, rifapentine, St John's Wort extract, apalutamide, lumacaftor, and ivosidenib. In some embodiments, the subject has not been administered a strong inducer of CYP3A4 within 14 days before the first regimen starts, i.e., before day 1 of cycle 1 of the first regimen.

In various embodiments, the subject is in further need of treatment with a CYP3A4 substrate. In some embodiments, the subject is not administered a CYP3A4 substrate in combination with sotorasib. Exemplary CYP3A4 substrates include, but are not limited to, abemaciclib, abiraterone, acalabrutinib, alectinib, alfentanil, alprazolam, amitriptyline, amlodipine, apixaban, aprepitant, aripiprazole, astemizole, atorvastatin, avanafil, axitinib, boceprevir, bosutinib, brexpiprazole, brigatinib, buspirone, cafergot, caffeine, carbamazepine, cariprazine, ceritinib, cerivastatin, chlorpheniramine, cilostazol, cisapride, citalopram, clarithromycin, clobazam, clopidogrel, cobimetinib, cocaine, codeine, colchicine, copanlisib, crizotinib, cyclosporine, dabrafenib, daclatasvir, dapsone, deflazacort, dexamethasone, dextromethorphan, diazepam, diltiazem, docetaxel, dolutegravir, domperidone, doxepin, elagolix, elbasvir/grazoprevir, eliglustat, enzalutamide, eplerenone, erythromycin, escitalopram, esomeprazole, estradiol, felodipine, fentanyl, finasteride, flibanserin, gleevec, haloperidol, hydrocortisone, ibrutinib, idelalisib, indacaterol, indinavir, irinotecan, isavuconazonium, ivabradine, ivacaftor, lansoprazole, lenvatinib, lercanidipine, lidocaine, linagliptin, lovastatin, macitentan, methadone, midazolam, naldemedine, naloxegol, nateglinide, nelfinavir, neratinib, netupitant/palonosetron, nevirapine, nifedipine, nisoldipine, nitrendipine, olaparib, omeprazole, ondansetron, osimertinib, ospemifene, palbociclib, panobinostat, pantoprazole, perampanel, pimavanserin, pimozide, pomalidomide, ponatinib, progesterone, propranolol, quetiapine, quinidine, quinine, regorafenib, ribociclib, rilpivirine, risperidone, ritonavir, rivaroxaban, roflumilast, rolapitant, romidepsin, ruxolitinib, salmeterol, saquinavir, selexipag, sildenafil, simeprevir, simvastatin, sirolimus, sonidegib, sorafenib, sunitinib, suvorexant, tacrolimus(fk506), tamoxifen, tasimelteon, taxol, telaprevir, telithromycin, terfenadine, testosterone, ticagrelor, tofacitinib, tolvaptan, torisel, tramadol, trazodone, valbenazine, vandetanib, velpatasvir, vemurafenib, venetoclax, venlafaxine, verapamil, vilazodone, vincristine, vorapaxar, voriconazole, zaleplon, and ziprasidone. See, e.g., Flockhart DA, Drug Interactions: Cytochrome P450 Drug Interaction Table. Indiana University School of Medicine (2007), https://drug-interactions.medicine.iu.edu, last accessed May 2021.

In some embodiments, the subject is not administered a CYP3A4 substrate in combination with sotorasib, wherein the CYP3A4 substrate is a CYP3A4 substrate with a narrow therapeutic index. Exemplary CYP3A4 substrates with a narrow therapeutic index include, but are not limited to, alfentanil, fentanyl, cyclosporine, pimozide, dihydroergotamine, quinidine, ergotamine, sirolimus, everolimus, and tacrolimus. In some embodiments, the subject has not been administered a CYP3A4 with a narrow therapeutic index within 14 days before the first regimen starts, i.e., before day 1 of cycle 1 of the first regimen.

In various embodiments, the subject is in further need of treatment with a P-glycoprotein (P-gp) substrate. In some embodiments, the subject is not administered a P-gp substrate in combination with sotorasib. Exemplary P-gp substrates include, but are not limited to dabigatran etexilate, digoxin, fexofenadine, everolimus, cyclosporine, sirolimus, and vincristine. See, e.g., www.fda.gov/drugs/drug-interactions-labeling/drug-development-and-drug-interactions-table-substrates-inhibitors-and-inducers, last accessed May 2021. In some embodiments, the subject is not administered a P-gp substrate in combination with sotorasib, wherein the P-gp substrate is a P-gp substrate with a narrow therapeutic index. Exemplary P-gp substrates with a narrow therapeutic index include, but are not limited to, digoxin, everolimus, cyclosporine, sirolimus, tacrolimus, and vincristine. P-gp substrates with a narrow therapeutic index are compounds for which minimal concentration changes may lead to serious toxicities. In some embodiments, the subject has not been administered a P-gp substrate with a narrow therapeutic index within 14 days before the first regimen starts, i.e., before day 1 of cycle 1 of the first regimen.

### Pembrolizumab

Pembrolizumab is the non-investigational product used in the Study of Example 2 below, as a comparator to sotorasib.

Pembrolizumab is a potent humanized immunoglobulin G4 monoclonal antibody (mAb) with high specificity of binding to the PD 1 receptor, thus inhibiting its interaction with PD-L1 and programmed cell death ligand-2 (PD-L2). Pembrolizumab is approved as a single agent for the front-line treatment of subjects with metastatic NSCLC expressing PD-L1 (TPS≥1%) as determined by an FDA-approved test, as well as after disease progression on or after platinum containing chemotherapy (see, e.g., KEYTRUDA® US Prescribing Information, Merck & Co., Kenilworth, NJ 07033 (revision 3/2023), which is herein incorporated by reference in its entirety). Subjects with EGFR or ALK genomic tumor aberrations should have disease progression on FDA approved targeted therapy for these aberrations before receiving pembrolizumab.

The comparator group in the study described in Example 2, the combination of pembrolizumab with carboplatin and pemetrexed, is a therapy of choice for subjects with nonsquamous NSCLC without actionable oncogenic driver mutations, e.g., for subjects with low or negative level of PD-L1 expression (NCCN Guidelines, 2022).

Pembrolizumab in combination with pemetrexed-platinum chemotherapy is approved based on results from cohort G of KEYNOTE-021 and the confirmatory KEYNOTE 189 study (Gandhi, et al., 2018; Langer et al., 2016). The initial results of KEYNOTE 189 demonstrated a significant improvement in OS, with an HR of 0.49 (95% CI: 0.38, 0.64; p<0.001) as well as in PFS with an HR of 0.52 (95% CI: 0.43, 0.64; p<0.001), and ORR of 47.6% in subjects treated with the immunotherapy chemotherapy combination versus 18.9% in the control group (p<0.001) (Gandhi et al., 2018; Gadgeel et al., 2020). As a result of these and other data, pembrolizumab is approved in the United States, the European Union, and other countries in combination with pemetrexed and platinum chemotherapy, as first line treatment of subjects with metastatic nonsquamous NSCLC, with no EGFR or ALK genomic tumor aberrations (see, e.g., KEYTRUDA® US Prescribing Information, Merck & Co., Kenilworth, NJ 07033 (revision 3/2023)). Thus, pembrolizumab with platinum doublet chemotherapy (the current standard of care) was selected as the study comparator.

### Patient Population

In various embodiments, the subject is an adult. In some embodiments, the subject is 18 years or older.

In various embodiments, the subject, prior to treatment with the methods provided herein, has not received a prior systemic anticancer therapy for the cancer. In some cases, the patient has not received a prior therapy for the KRAS G12C mutated cancer, e.g., metastatic colorectal cancer and pancreatic cancer. In some embodiments, the patient has not previously received therapy with an anti-angiogenic agent. In some embodiments, that patient is not concurrently being treated with an anti-angiogenic agent. In some embodiments, the anti-angiogenic agent is an anti-VEGF antibody (e.g., bevacizumab or ramucirumab), aflibercept, or regorafenib. In some embodiments, patient is not concurrently being treated with bevacizumab. In some embodiments, the patient has not received prior therapy for metastatic disease. The methods disclosed herein are front-line or first line treatments.

In various embodiments, the subject has a creatinine clearance (CrCl) of equal or greater than 45 mL/min as calculated by the Cockcroft-Gault formula. See Cockcroft et al., 1976. In various embodiments, the subject has a creatinine clearance (CrCl) equal or greater than 45 mL/min and equal or less than 125 mL/min.

In various embodiments, the subject has an Eastern Cooperative Oncology Group (ECOG) Performance Status of 0 or 1. See, e.g., Oken et al., 1982. Status 0 indicates fully active and able to carry on all pre-disease performance without restriction. Status 1 indicates restricted in physically strenuous activity but ambulatory and able to carry out work of a light or sedentary nature. Status 2 indicates ambulatory and capable of all selfcare but unable to carry out any work activities; up and about more than 50% of waking hours. Status 3 indicates capable of only limited selfcare, confined to bed or chair more than 50% of waking hours. Status 4 indicates completely disabled, cannot carry on any selfcare and totally confined to bed or chair. Status 5 indicates death.

### KRAS G12C Mutated Cancers

Provided herein are methods of treating cancer comprising a KRAS G12C mutation in a subject in need thereof comprising administering to the subject (a) a first regimen comprising (i) a therapeutically effective amount of sotorasib, (ii) a therapeutically effective amount of carboplatin and (iii) a therapeutically effective amount of pemetrexed and then (b) a second regimen comprising (i) the therapeutically effective amount of sotorasib and (ii) the therapeutically effective amount of pemetrexed. In various embodiments, the therapeutically effective amount of sotorasib is 240 mg. In various embodiments, the therapeutically effective amount of sotorasib is 960 mg.

Further, provided herein are methods of treating non-small cell lung cancer comprising a KRAS G12C mutation in a subject in need thereof comprising administering to the subject (a) a first regimen comprising (i) 240 mg of sotorasib, (ii) a therapeutically effective amount of carboplatin and (iii) a therapeutically effective amount of pemetrexed and then (b) a second regimen comprising (i) the 240 mg of sotorasib and (ii) the therapeutically effective amount of pemetrexed; wherein (1) the therapeutically effective amount of carboplatin is an amount corresponding to 5 mg/(mL×min) (AUC 5) multiplied with the subject's glomerular filtration rate (GFR) in mL/min increased by 25 mL/min, and wherein (2) the therapeutically effective amount of pemetrexed is 500 mg/m², and wherein (3) the subject, prior to treatment, has not received a prior systemic anticancer therapy for the cancer (first line treatment), and wherein (4) the cancer exhibits (a) a tumor cell (TC) score of less than 1% or (b) a PD-L1 tumor proportion score (TPS) of less than 1%.

Furthermore, provided herein are methods of treating non-small cell lung cancer comprising a KRAS G12C mutation in a subject in need thereof comprising administering to the subject (a) a first regimen comprising (i) 960 mg of sotorasib, (ii) a therapeutically effective amount of carboplatin and (iii) a therapeutically effective amount of pemetrexed and then (b) a second regimen comprising (i) the 960 mg of sotorasib and (ii) the therapeutically effective amount of pemetrexed; wherein (1) the therapeutically effective amount of carboplatin is an amount corresponding to 5 mg/(mL×min) (AUC 5) multiplied with the subject's glomerular filtration rate (GFR) in mL/min increased by 25 mL/min, and wherein (2) the therapeutically effective amount of pemetrexed is 500 mg/m², and wherein (3) the subject, prior to treatment, has not received a prior systemic anticancer therapy for the cancer (first line treatment), and wherein (4) the cancer exhibits (a) a tumor cell (TC) score of less than 1% or (b) a PD-L1 tumor proportion score (TPS) of less than 1%.

Without wishing to be bound by any particular theory and as previously discussed, the following is noted: sotorasib is a small molecule that specifically and irreversibly inhibits KRAS^{G12C} (Hong et al., 2020). Hong et al. report that “[p]reclinical studies showed that [sotorasib] inhibited nearly all detectable phosphorylation of extracellular signal-regulated kinase (ERK), a key down-stream effector of KRAS, leading to durable complete tumor regression in mice bearing KRAS p.G12C tumors.” (id., see a/so Canon et al., 2019, and Lanman et al., 2020).

Sotorasib was evaluated in a Phase 1 dose escalation and expansion trial with 129 subjects having histologically confirmed, locally advanced or metastatic cancer with the KRAS G12C mutation identified by local molecular testing on tumor tissues, including 59 subjects with non-small cell lung cancer, 42 subjects with colorectal cancer, and 28 subjects with other tumor types (Hong et al., 2020, at page 1208-1209). Hong et al. report a disease control rate (95% CI) of 88.1% for non-small cell lung cancer, 73.8% for colorectal cancer and 75.0% for other tumor types (Hong et al., 2020, at page 1213, Table 3). The cancer types showing either stable disease (SD) or partial response (PR) as reported by Hong et al. were non-small cell lung cancer, colorectal cancer, pancreatic cancer, appendiceal cancer, endometrial cancer, cancer of unknown primary, ampullary cancer, gastric cancer, small bowel cancer, sinonasal cancer, bile duct cancer, or melanoma (Hong et al., 2020, at page 1212 (Figure A), and Supplementary Appendix (page 59 (Figure S5) and page 63 (Figure S6)).

KRAS G12C mutations occur with the alteration frequencies shown in the table below (Cerami et al., 2012; Gao et al., 2013). For example, the table shows that 11.6% of subjects with non-small cell lung cancer have a cancer comprising a KRAS G12C mutation. Accordingly, sotorasib, which specifically and irreversibly binds to KRAS^{G12C} is useful for treatment of subjects having a cancer, including, but not limited to the following cancers:

| | Alteration |
|---|---|
| Cancer Type | Frequency |
| Non-Small Cell Lung Cancer | 11.6 |
| Small Bowel Cancer | 4.2 |
| Appendiceal Cancer | 3.6 |
| Colorectal Cancer | 3.0 |
| Cancer of Unknown Primary | 2.9 |
| Endometrial Cancer | 1.3 |
| Mixed Cancer Types | 1.2 |
| Pancreatic Cancer | 1.0 |
| Hepatobiliary Cancer | 0.7 |
| Small Cell Lung Cancer | 0.7 |
| Cervical Cancer | 0.7 |
| Germ Cell Tumor | 0.6 |
| Ovarian Cancer | 0.5 |
| Gastrointestinal Neuroendocrine | 0.4 |
| Tumor | |
| Bladder Cancer | 0.4 |
| Myelodysplastic/Myeloproliferative | 0.3 |
| Neoplasms | |
| Head and Neck Cancer | 0.3 |
| Esophagogastric Cancer | 0.2 |
| Soft Tissue Sarcoma | 0.2 |
| Mesothelioma | 0.2 |
| Thyroid Cancer | 0.1 |
| Leukemia | 0.1 |
| Melanoma | 0.1 |

In various embodiments, the cancer is a solid tumor. In various embodiments, the cancer is non-small cell lung cancer, small bowel cancer, appendiceal cancer, colorectal cancer, cancer of unknown primary, endometrial cancer, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, ampullary cancer, gastric cancer, sinonasal cancer, or bile duct cancer. In some embodiments, the cancer is non-small cell lung cancer, small bowel cancer, appendiceal cancer, colorectal cancer, cancer of unknown primary, endometrial cancer, pancreatic cancer, melanoma, ampullary cancer, gastric cancer, sinonasal cancer, or bile duct cancer. In various embodiments, the cancer is colorectal cancer. In some embodiments, the cancer is pancreatic cancer.

### Non-Small Cell Lung Cancer

Lung cancer is the leading cause of cancer death, with more than 80% of all lung cancer cases classified as NSCLC. Worldwide, lung cancer (small cell and non-small cell) is the second most common cancer overall, with an estimated 2.21 million cases in 2020 (World Health Organization Statistics, 2020). In 2020, more than 253537, 477543, and 1315136 new cases of lung cancer were reported in North America, Europe, and Asia, respectively. The estimated number of deaths from lung cancer in 2020 was 159641 in North America, 384176 in Europe, and 1112517 in Asia (Globocan—Lung Cancer, 2020). Advanced NSCLC (stage IllB/C and IV) is a serious and life-threatening disease, with a 5-year survival rate of only 9.3% (Surveillance, Epidemiology, and End Results [SEER], 2022).

Choice of systemic therapy for subjects with advanced stage NSCLC is based on the molecular characteristics of the cancer, specifically, presence of actionable driver genetic alterations and the level of expression of PD-L1 in tumor cells (NCCN Guidelines, 2022; Besse et al., 2014).

Level of expression of PD-L1 on tumor cells measured by TPS or tumor cell (TC) score correlates positively with the response rate and duration of the response to programmed cell death protein 1/ligand-1 (PD-1/L1) checkpoint inhibitors alone or in combination with platinum doublet chemotherapy in subjects with NSCLC without actionable mutations in EGFR or ALK genes (Reck et al., 2019; Gandhi et al., 2018; Paz-Ares et al., 2018). Combination of immune checkpoint inhibitors, such as pembrolizumab, with platinum doublet chemotherapy is a treatment of choice for advanced NSCLC negative for PD-L1 expression and without actionable mutations in EGFR or ALK genes.

In contrast, presence of actionable driver mutation in either EGFR or ALK gene signify low efficacy of PD-1/L1 checkpoint inhibitors, regardless of level of PD-L1 expression in the tumor. Hence, oral tyrosine kinase inhibitors (TKIs) of EGFR or ALK protein are an NCCN category 1 front-line therapy choice in such subjects. Role of immune checkpoint inhibitors in these groups of subjects is limited and it is usually combined with chemotherapy, commonly used beyond second line (NCCN Guidelines, 2022).

Subjects with advanced nonsquamous NSCLC without EGFR or ALKdriver mutations are treated with either pembrolizumab alone or cemiplimab-rwlc alone or immunotherapy (pembrolizumab, atezolizumab, or cemiplimab) in combination with platinum chemotherapy (Reck et al., 2019; Gandhi et al., 2018; Sezer et al., 2021; Gogishvili et al., 2022).

The efficacy of pembrolizumab in combination with pemetrexed and platinum chemotherapy was investigated in a randomized, multicenter, double-blind study in subjects with metastatic nonsquamous NSCLC, stratified by PD-L1 tumor expression levels, who had not previously received systemic therapy for metastatic disease and in whom there were no EGFR or ALK genomic tumor aberrations (KEYNOTE-189). Despite substantial improvement in 2-year overall survival (OS) in the intent-to-treat population in KEYNOTE-189 (45.5%; hazard ratio [HR]0.56), response rate in the pembrolizumab-chemotherapy group was only 48% (95% CI 43.1-53.0) (Gadgeel et al, 2020). Response rate, PFS, and OS were strongly associated with higher levels of PD-L1 expression in the tumor, as demonstrated in the updated analysis (Rodriguez-Abreu et al., 2021). Specifically, the objective response rate (ORR), PFS, and OS in the PD-L1<1% group were 33.1% (95% CI: 25.0, 42.0), 6.2 months (HR 0.67, 95% CI: 0.49, 0.93), and 17.2 months (HR 0.51, 95% CI: 0.36, 0.71) in comparison with 50% (95% CI: 41.0, 59.0), 9.4 months (HR 0.53, 95% CI: 0.38, 0.74), and 21.8 months (HR 0.66, 95% CI: 0.46, 0.96) in the PD-L1 1% to 49% group, and 62% (95% CI: 53.3, 70.4), 11.1 months (HR 0.35, 95% CI: 0.25, 0.49), and 27.7 months (HR 0.59, 95% CI: 0.40, 0.86) in the PD-L1>50% group. Similar observations have also been seen with cemiplimab in combination with carboplatin-pemetrexed (Gogishvili et al., 2022), suggesting that PD-L1 negative NSCLC subjects may benefit from alternative therapies and/or that additional biomarkers may help to further select subjects in this group who will benefit from a novel therapeutic option.

Over the past 2 decades, numerous driver mutations in specific genes were identified (i.e., EGFR, ERBB2, BRAF, ROS1, ALK, METex14 skipping, NTRK, and RET). Among subjects with driver mutations, improved outcomes have been observed for subjects treated with targeted therapies compared with those who did not receive targeted treatment (Kris et al., 2017). Efficacy of immunotherapy, especially in front-line settings, in the subjects with most driver mutations is lower than in subjects with NSCLC without driver mutations, and in some groups, specifically in ALK- and EGFR-driven cancers, the response rate of immune checkpoint inhibitors is low (Addeo et al., 2021).

The combination of targeted small molecule therapies (e.g., EGFR inhibitors, BRAF inhibitors, or vascular endothelial growth factor inhibitors) and immunotherapy agents (e.g., anti-PD[L]-1, anti-cytotoxic T-lymphocyte-associated protein 4 [CTLA-4]) has led to an unexpected increase in frequency and/or severity of adverse events. Some examples include liver enzyme elevations with or without concomitant bilirubin elevation (ipilimumab and vemurafenib, durvalumab and gefitinib, pembrolizumab and axitinib, durvalumab and osimertinib, pembrolizumab and gefitinib) (Yang et al., 2019; Rini et al., 2019; Ahn et al., 2016; Gibbons et al., 2016; Ribas et al., 2013), and interstitial lung disease (osimertinib and durvalumab) (Ahn et al., 2016). Increased frequency and degree of immune-mediated toxicities were noted even if oral targeted agents were administered subsequent to immunotherapy, likely due to lingering presence and effect of anti-PD-1/L1 therapeutic antibodies.

Hence, NCCN and ESMO recommends use of targeted therapies as a front-line treatment for subjects with advanced or metastatic NSCLC possessing targetable mutations (NCCN Guidelines, 2022; Hendriks et al, 2023a; Hendricks et al, 2023b). If used in driver-mutation positive NSCLC, immune checkpoint inhibitors are recommended only as a salvage therapy in combination with chemotherapy and after failure of available targeted agents.

Subjects with KRAS mutations, commonly associated with heavy smoking history, demonstrate similar response to immune-checkpoint inhibitors chemotherapy combination as subjects without KRAS mutation (Jeanson et al, 2019). Currently, NSCLC subjects with the KRAS G12C mutation are, like subjects without other molecularly defined targets, receiving anti-PD-1 inhibitors with or without chemotherapy in front-line therapy. Thus, subjects with previously untreated locally advanced or metastatic KRAS G12C mutant NSCLC treated with front-line targeted therapy, notably subjects whose tumors had <10% of tumor cells that expressed PD-L1 (Herbst et al., 2019; Gadgeel et al., 2019) represent a population in need of new treatment methods, such as the methods disclosed herein.

Accordingly, provided herein are methods for treating cancer comprising a KRAS G12C mutation in a subject in need thereof, wherein the cancer is non-small cell lung cancer. In various embodiments, the cancer is metastatic or locally advanced non-small cell lung cancer. In some embodiments the cancer is nonsquamous non-small cell lung cancer. In some embodiments, the cancer is nonsquamous non-small cell lung cancer of stage IV or advanced stage IIIB/C. See, e.g., AJCC Cancer Staging Manual 8^{th} ed., 2017.

### Determining KRAS G12C Mutation Status

Methods and tests for determining whether a subject has a cancer comprising a KRAS G12C mutation are known in the art. The determination of KRAS G12C mutation status can be performed using Guardant360® NGS (plasma) and Qiagen Therascreen® KRAS RGQ PCR (tissue; from formalin-fixed paraffin embedded (FFPE) tissue or cell block). KRAS G12C mutation status of a cancer can be determined with any test approved by a regulatory authority, such as the US Food and Drug Administration (FDA).

### Determining PD-L1 Expression Status

By way of background, PD-L1 is a transmembrane protein that downregulates immune responses through binding to its two receptors, programmed death-1 (PD-1) and B7-1. Keir et al., 2008. PD-1 is an inhibitory receptor expressed on T-cells following T-cell activation, which is sustained in states of chronic stimulation such as in chronic infection or cancer. Blank et al., 2007. Binding of PD-L1 with PD-1 inhibits T-cell proliferation, cytokine production, and cytolytic activity, leading to the functional inactivation or exhaustion of T-cells. Blank et al. 2007. B7.1 is a molecule expressed on antigen presenting cells and activated T-cells. PD-L1 binding to B7.1 on T-cells and antigen presenting cells can mediate downregulation of immune responses, including inhibition of T-cell activation and cytokine production. Butte et al., 2007. PD-L1 expression has been observed in immune cells and malignant cells (Dong et al., 1999; Massard et al., 2016) and aberrant expression of PD-L1 on malignant cells has been reported to impede anti-tumor immunity, resulting in immune evasion. Blank et al., 2007; Massard et al., 2016. Therefore, interruption of the PD-L1/PD-1 pathway represents an attractive strategy to reinvigorate tumor-specific T-cell immunity suppressed by the expression of PD-L1 in the tumor microenvironment. The association between PD-L1 expression in TC or tumor-infiltrating immune cells (IC) and clinical benefit with PD-L1/PD-1 pathway inhibitors has been reported across multiple cancers. See a/so, e.g., VENTANA PD-L1 (SP263) Assay, Instructions 2021-10-10, 1020514US Rev 1, Ventana Medical Systems, Inc. 1910 E. Innovation Park Drive Tucson, Arizona 85755 USA, available at https://www.accessdata.fda.gov/cdrh_docs/pdfi6/P160046S010C.pdf, last accessed Mar. 31, 2023.

PD-L1 expression can be determined by methods known in the art. For example, PD-L1 expression can be detected using PD-L1 IHC 22C3 pharmDx, an FDA-approved in vitro diagnostic immunohistochemistry (IHC) test developed by Dako and Merck as a companion test for treatment with pembrolizumab. See, PD-L1 IHC 22C3 pharmDx, Dako North America, Inc. 6392 Via Real, Carpinteria, California 93013, USA, available at https://www.accessdata.fda.gov/cdrh_docs/pdfi5/p150013c.pdf, Edition 09/15, last accessed Mar. 31, 2023, which is incorporated herewith by reference in its entirety. PD-L1 IHC 22C3 pharmDx is a qualitative immunohistochemical assay using Monoclonal Mouse Anti-PD-L1, Clone 22C3 intended for use in the detection of PD-L1 protein in formalin-fixed, paraffin-embedded (FFPE) non-small cell lung cancer (NSCLC) tissue using EnVision FLEX visualization system on Autostainer Link 48. PD-L1 protein expression is determined by using Tumor Proportion Score (TPS), which is the percentage of viable tumor cells showing partial or complete membrane staining.

For example, PD-L1 expression can also be detected using Ventana SP263 assay (developed by Ventana in collaboration with AstraZeneca). This assay is a qualitative immunohistochemistry assay using rabbit monoclonal anti-PD-L1 clone SP263 intended for use in the assessment of the programmed death ligand-1 (PD-L1) protein in formalin-fixed, paraffin-embedded (FFPE) non-small cell lung carcinoma (NSCLC) tissue specimens by light microscopy. The assay is used with the OptiView DAB IHC Detection Kit for staining on the BenchMark ULTRA instrument. PD-L1 protein expression in NSCLC is determined by the percentage of tumor cells (% TC) with any membrane staining above background. The assay is indicated as an aid in identifying subjects eligible for treatment with, e.g., atezolizumab (TECENTRIQ®) for non-small cell lung cancer with a TC score of ≥1%. See, VENTANA PD-L1 (SP263) Assay, Instructions 2021-10-10, 1020514US Rev 1, Ventana Medical Systems, Inc. 1910 E. Innovation Park Drive Tucson, Arizona 85755 USA, available at https://www.accessdata.fda.gov/cdrh_docs/pdfi6/P160046S010C.pdf, last accessed Mar. 31, 2023, which is incorporated herewith by reference in its entirety.

In some embodiments, a test approved by a regulatory authority, such as the US Food and Drug Administration (FDA), is used to determine the PD-L1 negativity by either using the PD-L1 TPS or TC score of a cancer as recited in the methods disclosed herein. In various embodiments, the PD-L1 TPS or TC score is determined using an immunohistochemistry (IHC) test. In various embodiments, the IHC test conducted with samples acquired by, for example, resection, core needle biopsy (CNB), or fine needle aspiration (FNA). In some embodiments, the IHC test is the PD-L1 IHC 22C3 pharmDx test to determine the TPS. In some embodiments, the IHC test is the Ventana PD-L1 (SP263) IHC assay to determine the TC score.

In various embodiments, the cancer exhibits a PD-L1 TPS of less than 100%, 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the cancer exhibits a PD-L1 TPS of less than 1%. In some embodiments, the cancer exhibits a PD-L1 TPS in the range of more than or equal to 0% and less than 1%. In some embodiments, the cancer exhibits a PD-L1 TPS score of 1-49%. In some embodiments, the cancer exhibits a PD-L1 TIPS score of 50% or greater (i.e., 50%-100%). In some embodiments, the PD-L1 TIPS scope is determined using the PD-L1 IHC 22C3 pharmDx test.

In various embodiments, the cancer exhibits a TC score of less than 100%, 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the cancer exhibits a TC score of less than 1%. In some embodiments, the cancer exhibits a TC score in the range of more than or equal to 0% and less than 1%. In some embodiments, the cancer exhibits a TC score of 1-49%. In some embodiments, the cancer exhibits a TC score of 50% or greater (i.e., 50%-100%). In some embodiments, the TC score is determined using the Ventana PD-L1 (SP263) IHC assay.

### Determining EGFR, ALK, ROS1 Alteration Status

Subjects with advanced non-small cell lung cancer (NSCLC) possessing actionable mutations in epithelial growth factor receptor (EGFR), anaplastic lymphoma kinase (ALK), ROS proto-oncogene 1 (ROS1), and a few other genes, demonstrate superior outcomes with small molecule targeted therapies in contrast with platinum doublet chemotherapy or immune checkpoint inhibitors alone or in combination with chemotherapy (European Society for Medical Oncology [ESMO] Clinical Practice Guidelines, 2020; National Comprehensive Cancer Network [NCCN]) Guidelines, 2022).

Accordingly, provided herein are methods for treating cancer, wherein the cancer does not comprise an EGFR alteration. In some embodiments, the cancer does not comprise an ALK alteration. In some embodiments, the cancer does not comprise a ROS1 alteration. In some embodiments, the cancer does not comprise an EGFR or ALK alteration.

Methods of determining EGFR, ALK, and ROS1 alteration status are known in the art (see, e.g., Oncomine Dx Target Test by ThermoFisher Scientific or https://testdirectory.questdiagnostics.com/test/test-guides/CF_NSCLC/non-small-cell-lung-cancer-nsclc-laboratory-support-of-diagnosis-and-management, last accessed Mar. 30, 2023).

### Embodiments

1. A method of treating cancer comprising a KRAS G12C mutation in a subject in need thereof, the method comprising: administering to the subject (a) a first regimen comprising (i) a therapeutically effective amount of sotorasib, (ii) a therapeutically effective amount of carboplatin and (iii) a therapeutically effective amount of pemetrexed and then administering (b) a second regimen comprising (i) the therapeutically effective amount of sotorasib and (ii) the therapeutically effective amount of pemetrexed.

2. The method of embodiment 1, wherein the first regimen is administered for a period of 4 cycles.

3. The method of embodiment 2, wherein each cycle is a period of 21 days.

4. The method of any one of embodiments 1 to 3, wherein the therapeutically effective amount of sotorasib is 240 mg.

5. The method of any one of embodiments 1 to 3, wherein the therapeutically effective amount of sotorasib is 960 mg.

6. The method of any one of embodiments 1 to 5, wherein the sotorasib is administered orally.

7. The method of any one of embodiments 1 to 6, wherein the sotorasib is administered once daily.

8. The method of any one of embodiments 1 to 7, wherein the sotorasib is administered as a solid dosage form.

9. The method of embodiment 8, wherein the solid dosage form is a tablet.

10. The method of embodiment 9, wherein the sotorasib is administered as one tablet comprising 240 mg of sotorasib, two tablets each comprising 120 mg of sotorasib, three tablets each comprising 320 mg of sotorasib, four tablets each comprising 240 mg of sotorasib, or eight tablets each comprising 120 mg of sotorasib.

11. The method of any one of embodiments 1 to 10, wherein the therapeutically effective amount of carboplatin is an amount corresponding to 5 mg/(mL×min) (AUC 5) multiplied with the subject's glomerular filtration rate (GFR) in mL/min increased by 25 mL/min.

12. The method of embodiment 11, wherein the therapeutically effective amount of carboplatin is 750 mg or less.

13. The method of any one of embodiments 1 to 12, wherein the carboplatin is administered intravenously.

14. The method of any one of embodiments 2 to 13, wherein the carboplatin is administered on day 1 of each cycle.

15. The method of any one of embodiments 1 to 14, wherein the subject is not administered carboplatin during the second regimen.

16. The method of any one of embodiments 1 to 15, wherein the therapeutically effective amount of pemetrexed is 500 mg/m².

17. The method of any one of embodiments 1 to 16, wherein pemetrexed is administered intravenously.

18. The method of any one of embodiments 1 to 17, wherein the administration of pemetrexed starts on day 1 of the first regimen.

19. The method of any one of embodiments 1 to 18, wherein the pemetrexed is administered in 21 day intervals during the first regimen and the second regimen.

20. The method of any one of embodiments 1 to 19, further comprising administering to the subject a therapeutically effective amount of folic acid.

21. The method of embodiment 20, wherein the therapeutically effective amount of folic acid is 350 μg to 1000 μg once daily.

22. The method of embodiment 20, wherein the therapeutically effective amount of folic acid is 400 μg once daily.

23. The method of embodiment 20, wherein the therapeutically effective amount of folic acid is 400 μg to 1000 μg once daily.

24. The method of embodiment 20 or embodiment 23, wherein the folic acid administration commences 7 days prior to pemetrexed administration and ends 21 days after cessation of pemetrexed administration.

25. The method of any one of embodiments 1 to 24, further comprising administering to the subject a therapeutically effective amount of vitamin B12.

26. The method of embodiment 25, wherein the therapeutically effective amount of vitamin B12 is 1 mg.

27. The method of embodiment 25 or embodiment 26, wherein the vitamin B12 is administered in the week prior to first pemetrexed administration and once every 9 weeks (±2 weeks) thereafter until cessation of pemetrexed administration.

28. The method of embodiment 25 or embodiment 26, wherein the vitamin B12 is administered one week prior to first pemetrexed administration and once every 9 weeks thereafter until cessation of pemetrexed administration.

29. The method of any one of embodiments 25 to 28, wherein the vitamin B12 is administered intramuscularly.

30. The method of any one of embodiments 1 to 29, further comprising administering to the subject a therapeutically effective amount of dexamethasone.

31. The method of embodiment 30, wherein the therapeutically effective amount of dexamethasone is 4 mg twice daily.

32. The method of embodiment 30 or embodiment 31, wherein the dexamethasone is administered the day before, the day of, and the day after each pemetrexed administration to the subject.

33. The method of any one of embodiments 1 to 32, wherein the subject is an adult.

34. The method of any one of embodiments 1 to 33, wherein the subject, prior to treatment, has not received a prior systemic anticancer therapy for the cancer (first line treatment).

35. The method of any one of embodiments 1 to 34, wherein the subject has a creatinine clearance (CrCl) of equal or greater than 45 mL/min as calculated by the Cockcroft-Gault formula.

36. The method of any one of embodiments 1 to 35, wherein the subject has an ECOG Performance Status of 0 or 1.

37. The method of any one of embodiments 1 to 36, wherein the cancer exhibits a TC score of less than 1%.

38. The method of embodiment 37, wherein the therapeutically effective amount of sotorasib is 960 mg.

39. The method of embodiment 37, wherein the TC score was determined using the Ventana PD-L1 (SP263) IHC assay.

40. The method of any one of embodiments 1 to 36, wherein the cancer exhibits a PD-L1 tumor proportion score (TPS) of less than 1%.

41. The method of embodiment 40, wherein the therapeutically effective amount of sotorasib is 960 mg.

42. The method of embodiment 40, wherein the TPS was determined using the PD-L1 IHC 22C3 pharmDx assay.

43. The method of any one of embodiments 1 to 42, wherein the cancer does not comprise an EGFR or ALK alteration.

44. The method of any one of embodiments 1 to 43, wherein the cancer is lung cancer.

45. The method of any one of embodiments 1 to 43, wherein the cancer is non-small cell lung cancer.

46. The method of any one of embodiments 1 to 43, wherein the cancer is nonsquamous non-small cell lung cancer.

47. The method of embodiment 46, wherein the nonsquamous non-small cell lung cancer is stage IV or advanced stage IIIB/C.

48. The method of any one of embodiments 1 to 47, wherein the patient is not concomitantly administered an antibody.

49. The method of embodiment 48, wherein the antibody is an anti-VEGF antibody.

50. The method of embodiment 49, wherein the anti-VEGF antibody is bevacizumab.

51. Sotorasib for use in treating a KRAS G12C mutated cancer wherein the treating comprises administering to the subject (a) a first regimen comprising (i) a therapeutically effective amount of sotorasib, (ii) a therapeutically effective amount of carboplatin and (iii) a therapeutically effective amount of pemetrexed and then administering to the subject (b) a second regimen comprising (i) the therapeutically effective amount of sotorasib and (ii) the therapeutically effective amount of pemetrexed.

52. Sotorasib for use in treating a KRAS G12C mutated cancer wherein the treating comprises administering to the subject (a) a first regimen comprising (i) sotorasib, (ii) carboplatin and (iii) pemetrexed and then administering to the subject (b) a second regimen comprising (i) sotorasib and (ii) pemetrexed.

### EXAMPLES

### Example 1—a Phase 1b/2, Protocol Evaluating the Safety, Tolerability, Pharmacokinetics, and Efficacy of Sotorasib Monotherapy and in Combination with Other Anti-Cancer Therapies in Subjects with Advanced Solid Tumors with KRAS p.G12C Mutation (CodeBreak 101)

### Study Design

Subprotocol F is part of Study 20190135 master protocol, Study 20190135, which evaluates various investigational combinations of sotorasib with other agents in advanced solid tumors with KRAS p.G12C mutation (CodeBreaK 101, ClinicalTrials.gov identifier: NCT04185883, https://clinicaltrials.gov/ct2/show/NCT04185883, last accessed Mar. 27, 2023). Cohort A of subprotocol F of CodeBreaK 101 evaluates the safety, tolerability, and efficacy of sotorasib in combination with carboplatin and pemetrexed for 4 cycles, followed by maintenance therapy with sotorasib plus pemetrexed.

The study includes a dose exploration phase (part 1) and expansion phase (part 2) and is being conducted at approximately 65 study centers in the US. Part 1 of the study consists of 2 cohorts. Cohorts A assess the safety of sotorasib in combination with carboplatin and pemetrexed (4 cycles (21 days each) followed by maintenance therapy with sotorasib and pemetrexed). Currently, subjects have been treated, inter alia, in cohort A part 1, cohort A1 part 2, and cohort A2 part 2 as listed below:

$$Cohort A part 1 = Sotorasib 960 mg QD + carboplatin AUC 5 Q 3 W + pemetrexed 500 mg / {m}^{2} Q 3 W ( n = 7 )$$

$$Cohort A 1 part 2 = Sotorasib 960 mg QD + carboplatin AUC 5 Q 3 W + pemetrexed 500 mg / {m}^{2} Q 3 W : Treatment Naive ( n = 11 )$$

$$Cohort A 2 part 2 = Sotorasib 960 mg QD + carboplatin AUC 5 Q 3 W + pemetrexed 500 mg / {m}^{2} Q 3 W : With History of Prior Therapy ( n = 8 )$$

Vitamin B12 and dexamethasone have been administered to trial subjects in accordance with pemetrexed label instructions (for the United States, see, e.g., ALIMTA® U.S. Prescribing Information, Lilly USA, LLC, Indianapolis, Indiana 46285 (revision 8/2022), which is herein incorporated by reference in its entirety). Primary safety endpoint was incidence of dose-limiting toxicities, treatment-emergent adverse events, treatment-related adverse events, and changes in vital signs, electrocardiograms, and clinical laboratory tests. Secondary efficacy endpoints included objective response rate (ORR), disease control rate (DCR), duration of response (DOR), time to response (TTR), overall survival (OS), progression-free survival (PFS), duration of stable disease and pharmacokinetic (PK) parameters.

The planned enrollment for sotorasib in combination with carboplatin and pemetrexed is up to 47 subjects.

### Subject Disposition and Demographics

As of the data snapshot date of 1 Dec. 2022, a total of 26 subjects were enrolled in cohort A subprotocol F of Study 20190135 and had received at least 1 dose of any investigational product. Of these 26 subjects, there were 17 men and 9 women. Subjects were White (n=23) or Black/African American (n=3) with a mean (SD) age of 65.3 (8.8) years. Level of PD-L1 expression was reported as expression on ≥50% of tumor cells in 7 of 26 subjects (26.9%), 10% to 49% in 4 of 26 subjects (15.4%), and expression on <1% tumor cells in 7 of 26 subjects (26.9%) (unknown in 8 of 26 subjects [30.8%]).

In cohort A part 1, 5 of 7 subjects (71.4%) had no prior history of anti-cancer therapy in the metastatic with 3 subjects (42.9%) without prior anti-PD-1/PD-L1 therapy. All subjects in cohort A1 part 2 were treatment naive and all subjects in cohort A2 part 2 had a history of prior anticancer therapy, including 5 of 8 subjects (62.5%) who received prior anti-PD-1/PD-L1 therapy.

### Exposure

In cohort A part 1, subjects had a median (min to max) exposure to sotorasib of 148.0 (93 to 589) days. Median sotorasib exposure in cohort A1 part 2 was 85.0 (9 to 191) days and in cohort A2 part 2 was 46.5 (7 to 233) days. Exposure to carboplatin ranged from median (min to max) of 60.5 (15 to 85) to 84.0 (63 to 91) days across cohorts and exposure to pemetrexed ranged from median (SD) of 70.5 (15 to 253) to 105.0 (16 to 197) days across cohorts.

### Efficacy Data

As of 1 Dec. 2022, 23 subjects in part A were evaluable for efficacy based on having received at least 1 dose of investigational products, had at least 1 measurable lesion at baseline assessed using RECIST 1.1 (Eisenhauer et al., 2009), and had the opportunity to be followed for at least 7 weeks. A summary of objective response by investigator assessments is shown by cohort in Table 1. For the analysis of best overall response (BOR), among these 23 subjects, 6 (26.1%) had a confirmed partial response (PR), 14 (60.9%) had stable disease (SD) (including 12 unconfirmed PR awaiting confirmatory scan), 1 (4.3%) had progressive disease (PD), and 2 subjects did not have post-baseline assessments. Confirmed ORR was 42.9% (95% CI: 9.90 to 81.59) in cohort A part 1, 11.1% (95% CI: 0.28 to 48.25) in cohort A1 part 2, and 28.6% (95% CI: 3.67 to 70.96) in cohort A2 part 2.

TABLE 1
Summary of Objective Response by Investigator Assessments (ORR Analysis Set—Study
20170543 Subprotocol F)

| | | Part 2 Cohort A1 | Part 2 Cohort A2 | |
|---|---|---|---|---|
| | | 960 mg | 960 mg | |
| | Part 1 Cohort A | Sotorasib + | Sotorasib + | Total |
| | 960 mg | Carboplatin + | Carboplatin | 960 mg |
| | Sotorasib + | Pemetrexed: | + Pemetrexed: | Sotorasib + |
| | Carboplatin + | Treatment | With History of | Carboplatin + |
| | Pemetrexed | Naive | Prior Therapy | Pemetrexed |
| | (N = 7) | (N = 9) | (N = 7) | (N = 23) |
| Best overall response-n (%) | | | | |
| Complete response (CR) | | | | |
| Confirmed | 0 (0.0) | 0 (0.0) | 0 (0.0) | 0 (0.0) |
| Confirmed and unconfirmed awaiting | 0 (0.0) | 0 (0.0) | 0 (0.0) | 0 (0.0) |
| confirmatory scan | | | | |
| Partial response (PR) | | | | |
| Confirmed | 3 (42.9) | 1 (11.1) | 2 (28.6) | 6 (26.1) |
| Confirmed and unconfirmed awaiting | 3 (42.9) | 6 (66.7) | 3 (42.9) | 12 (52.2) |
| confirmatory scan | | | | |
| Stable disease (SD) | 4 (57.1) | 8 (88.9) | 2 (28.6) | 14 (60.9) |
| Progressive disease (PD) | 0 (0.0) | 0 (0.0) | 1 (14.3) | 1 (4.3) |
| Not evaluable (NE) | 0 (0.0) | 0 (0.0) | 0 (0.0) | 0 (0.0) |
| Not done | 0 (0.0) | 0 (0.0) | 2 (28.6) | 2 (8.7) |
| Objective response rate (ORR) | | | | |
| Confirmed-n (%) | 3 (42.9) | 1 (11.1) | 2 (28.6) | 6 (26.1) |
| 95% CI^{a} | (9.90, 81.59) | (0.28, 48.25) | (3.67, 70.96) | (10.23, 48.41) |
| Confirmed and unconfirmed awaiting | 3 (42.9) | 6 (66.7) | 3 (42.9) | 12 (52.2) |
| confirmatory scan-n (%) | | | | |
| 95% CI^{a} | (9.90, 81.59) | (29.93, 92.51) | (9.90, 81.59) | (30.59, 73.18) |

N = Number of subjects in the analysis set.
n = Number of subjects with observed data.
CI = Confidence Interval.
Subjects who are included in the ‘Confirmed and unconfirmed awaiting confirmatory scan’ category may also have their BOR summarized in one of the other categories.
^{a}Exact 95% confidence interval was calculated using the Clopper Pearson method
ORR analysis set includes all subjects who received at least 1 dose of investigational products, have one or more measurable lesions at baseline assessed using RECIST 1.1 and have the opportunity to be followed for at least 7 weeks starting from day 1. One subject in Part 2 Cohort A1 had a BOR of SD based on their first post-baseline scan but is not included in ORR Analysis Set as they had not had the opportunity to be followed for at least 7 weeks starting from day 1 at the time of data cutoff.
Data cutoff date: 01DEC2022.

### Safety Data

As of 1 Dec. 2022, 26 subjects had received at least 1 investigational product and were evaluable for safety. A summary of treatment-emergent adverse events is provided in Table 2. Twenty-four subjects (92.3%) had at least 1 treatment-emergent adverse event. The most common adverse events, occurring in ≥50% of subjects per cohort were as follows:
Cohort A part 1 (n=7): diarrhea (71.4%), nausea (71.4%), fatigue (71.4%), and anemia (57.1%) in cohort A part 1
Cohort A1 part 2 (n=11): none in cohort A1 part 2
Cohort A2 part 2 (n=8): decreased appetite (50.0%) in cohort A2 part 2

Overall, 12 of 26 subjects (46.2%) had 1 or more treatment-emergent adverse events that were Common Terminology Criteria for Adverse Events (CTCAE) v5 grade 3, including anemia (n=5), diarrhea (n=2), abdominal pain, alanine aminotransferase increased, aspartate aminotransferase increased, blood alkaline phosphatase (ALP) increased, coronavirus disease 2019 (COVID-19), decreased appetite, dental caries, febrile neutropenia, hepatic function abnormal, hepatotoxicity, hypertransaminasaemia, nausea, neutropenia, neutrophil count decreased, platelet count decreased, proctalgia, pulmonary embolism (n=1, each). Three subjects had a grade 4 treatment-emergent adverse event of neutropenia (n=2), neutrophil count decreased, and thrombocytopenia (n=1, each). There were no fatal treatment-emergent adverse events.

Twenty-four subjects (92.3%) had at least 1 treatment-emergent adverse event considered to be related to study treatment. Six of 26 subjects (23.1%) had 1 or more serious adverse events, and 3 of 26 subjects (11.5%) discontinued sotorasib due to an adverse event. In general, the reported adverse events were consistent with the individual safety profiles of the investigation products and no new safety concerns have been identified.

TABLE 2
Summary of Treatment-emergent Adverse Events (Safety Analysis Set-Subprotocol F).

| | | | Part 2 Cohort A2 | | |
|---|---|---|---|---|---|
| | | Part 2 Cohort | 960 mg | | |
| | Part 1 Cohort | A1 | Sotorasib + | | |
| | A | 960 mg | Carboplatin + | Part 2 Total | |
| | 960 mg | Sotorasib + | Pemetrexed: | 960 mg | Total 960 mg |
| | Sotorasib + | Carboplatin + | With History | Sotorasib + | Sotorasib + |
| | Carboplatin + | Pemetrexed: | of Prior | Carboplatin + | Carboplatin + |
| | Pemetrexed | Treatment Naive | Therapy | Pemetrexed | Pemetrexed |
| | (N = 7) | (N = 11) | (N = 8) | (N = 19) | (N = 26) |
| | n (%) | n (%) | n (%) | n (%) | n (%) |
| All treatment-emergent adverse | 7 (100.0) | 9 (81.8) | 8 (100.0) | 17 (89.5) | 24 (92.3) |
| events | | | | | |
| Fatal adverse events | 0 (0.0) | 0 (0.0) | 0 (0.0) | 0 (0.0) | 0 (0.0) |
| Grade ≥ 2 | 7 (100.0) | 7 (63.6) | 7 (87.5) | 14 (73.7) | 21 (80.8) |
| Grade ≥ 3 | 7 (100.0) | 3 (27.3) | 5 (62.5) | 8 (42.1) | 15 (57.7) |
| Grade ≥ 4 | 1 (14.3) | 0 (0.0) | 2 (25.0) | 2 (10.5) | 3 (11.5) |
| Serious adverse events | 2 (28.6) | 2 (18.2) | 2 (25.0) | 4 (21.1) | 6 (23.1) |
| Leading to discontinuation of | 3 (42.9) | 0 (0.0) | 0 (0.0) | 0 (0.0) | 3 (11.5) |
| sotorasib | | | | | |
| Leading to discontinuation of | 2 (28.6) | 0 (0.0) | 0 (0.0) | 0 (0.0) | 2 (7.7) |
| carboplatin | | | | | |
| Leading to discontinuation of | 3 (42.9) | 0 (0.0) | 0 (0.0) | 0 (0.0) | 3 (11.5) |
| pemetrexed | | | | | |
| Leading to discontinuation of | — | — | — | — | — |
| docetaxel | | | | | |
| Any treatment-related treatment- | 7 (100.0) | 9 (81.8) | 8 (100.0) | 17 (89.5) | 24 (92.3) |
| emergent adverse events | | | | | |
| Fatal adverse events | 0 (0.0) | 0 (0.0) | 0 (0.0) | 0 (0.0) | 0 (0.0) |
| Grade ≥ 2 | 6 (85.7) | 6 (54.5) | 7 (87.5) | 13 (68.4) | 19 (73.1) |
| Grade ≥ 3 | 6 (85.7) | 3 (27.3) | 5 (62.5) | 8 (42.1) | 14 (53.8) |
| Grade ≥ 4 | 1 (14.3) | 0 (0.0) | 2 (25.0) | 2 (10.5) | 3 (11.5) |
| Serious adverse events | 2 (28.6) | 1 (9.1) | 2 (25.0) | 3 (15.8) | 5 (19.2) |

CRF = case report form;
CTCAE = Common Terminology Criteria for Adverse Events;
eCRF = electronic case report form;
MedDRA = Medical Dictionary for Regulatory Activities;
N = Number of subjects in the analysis set.
n = Number of subjects with observed data.
Treatment-emergent adverse events are Adverse Events (AEs) starting on or after the first dose of investigational product as determined by the flag indicating if the adverse event started prior to the first dose on the Events CRF and up to and including 30 days after the last dose of investigational product or the End of Study date, whichever is earlier.
A treatment-related AE is any treatment-emergent AE with the relationship flag on the Events eCRF indicating there is a reasonable possibility that the event may have been caused by investigational medicinal product. In the unlikely event that the relationship is missing, the treatment-emergent event is considered treatment-related.
For subjects with multiple events under the same category, only the worst grade is reported.
Coded using MedDRA version 25.1. Severity graded using CTCAE version 5.0.
Data cutoff date: 01DEC2022

### Updated Data Cut Dated 3 Feb. 2023

As of 3 Feb. 2023, 30 subjects (median age, 67 years; 60% male; ECOG 0/1: 47%/53%) were treated with sotorasib in combination with carboplatin and pemetrexed. 19 subjects were treatment-naive (Part 2 Cohort A1, first line (1 L)) and 11 had prior treatment (Part 2 Cohort A2, second and higher line (2L+)) in the metastatic/locally advanced setting. Treatment-related adverse events (TRAEs) occurred in 29 (97%) subjects, being grade 3-4 in 19 (63%) subjects (Table 3). The most common grade 3-4 TRAEswere neutropenia (9 [30%]), anemia and thrombocytopenia (both 5 subjects [17%]). No fatal adverse events occurred. In the first line setting (1C), in which treatment naive subjects were treated with sotorasib in combination with carboplatin and pemetrexed, the ORR (objective response rate, confirmed+unconfirmed) was 73% (95% CI: 45, 92), with 100% DCR (disease control rate, Table 3). For subjects with <1% PD-L1 tumor proportion score (TPS) treated in 1L, responses occurred in 8 of 11 subjects (ORR 73%). In the second line setting (2L+), ORR was 55%. Among all confirmed responders, median DOR (duration of response) was not yet estimable. PFS and OS data were immature.

The combination of sotorasib with carboplatin and pemetrexed was safe and tolerable, with incidences of grade 3-4 TRAEs largely consistent with other platinum doublet-based approaches. Promising efficacy was observed, most notably in treatment-naive subjects with low PD-L1 expression who have a high unmet need.

TABLE 3

| | Part 2 | Part 2 | |
|---|---|---|---|
| | Cohort A1 | Cohort A2 | |
| | 960 mg | 960 mg | |
| | Sotorasib + | Sotorasib + | Part 2I |
| | Carboplatin + | Carboplatin + | 960 mg |
| | Pemetrexed: | Pemetrexed: | Sotorasib + |
| | Treatment | With History | Carboplatin + |
| | Naive | of Prior | Pemetrexed |
| | (1L) | Therapy (2L+) | Total |
| Safety, n (%) | N = 19 | N = 11 | N = 30 |
| TRAE, any grade | 18 (95) | 11 (100) | 29 (97) |
| Most common (≥25% | | | |
| overall) TRAEs, any grade | | | |
| Neutropenia^{a} | 9 (47) | 6 (55) | 15 (50) |
| Thrombocytopenia^{b} | 7 (37) | 4 (36) | 11 (37) |
| Anemia | 7 (37) | 4 (36) | 11 (37) |
| ALT increased | 5 (26) | 5 (45) | 10 (33) |
| Asthenia | 5 (26) | 4 (36) | 9 (30) |
| Decreased appetite | 5 (26) | 4 (36) | 9 (30) |
| Diarrhea | 7 (37) | 2 (18) | 9 (30) |
| Vomiting | 7 (37) | 2 (18) | 9 (30) |
| AST increased | 4 (21) | 4 (36) | 8 (27) |
| Nausea | 5 (26) | 3 (27) | 8 (27) |
| TRAE, grade 3-4 | 10 (53) | 9 (82) | 19 (63) |
| Most common (≥5% | | | |
| overall) TRAE, grade 3-4 | | | |
| Neutropenia^{a} | 4 (21) | 5 (45) | 9 (30) |
| Anemia | 4 (21) | 1 (9) | 5 (17) |
| Thrombocytopenia^{b} | 3 (16) | 2 (18) | 5 (17) |
| Febrile neutropenia | 1 (5) | 1 (9) | 2 (7) |
| ALT increased | 0 | 2 (18) | 2 (7) |
| Diarrhea | 2 (11) | 0 | 2 (7) |
| Nausea | 2 (11) | 0 | 2 (7) |
| TRAE leading to | 3 (16) | 1 (9) | 4 (13) |
| discontinuation | | | |
| of sotorasib | | | |
| TRAE leading to | 2 (11) | 1 (9) | 3 (10) |
| discontinuation of | | | |
| carboplatin | | | |
| TRAE leading to | 4 (21) | 1 (9) | 5 (17) |
| discontinuation of | | | |
| pemetrexed | | | |
| Efficacy^{c}, % (95% CI) | N = 15 | N = 11 | N = 26 |
| ORR, confirmed | 60 (32, 84) | 46 (17, 77) | 54 (33, 73) |
| ORR, | 73 (45, 92) ^{d} | 55 (23, 83) ^{e} | 65 (44, 83) |
| confirmed + unconfirmed | | | |
| DCR | 100 (78, 100) | 82 (48, 98) | 92 (75, 99) |

TRAE = Treatment-Related Adverse Effects;
ORR = Objective Response Rate;
DCR = Disease Control Rate.
^{a}Includes reported TRAEs of neutropenia and neutrophil count decreased.
^{b}Includes reported TRAEs of thrombocytopenia and platelet count decreased.
^{c}Includes all pts who received at least 1 dose of investigational products, have ≥1 measurable lesion at baseline per RECIST 1.1 and have opportunity to be followed for ≥7 weeks.
^{d} ORR based on 9-confirmed partial response (PR), 2-unconfirmed PR; remaining best overall responses (BORs) were 4-stable disease (SD).
^{e} ORR based on 1-unconfirmed complete response (CR), 5-confirmed PR; remaining BORs were 3-SD, 1-progressive disease (PD), 1-not done.

### Updated Data Cut Dated 7 May 2023

As of 7 May 2023, 38 subjects were treated with sotorasib in combination with carboplatin and pemetrexed.

### Baseline Characteristics

Patient enrollment cohorts were Part 1 Cohort A (prior anti-PD-1/L1 immunotherapy and/or platinum-based combination chemotherapy, or refused standard therapy), Part 2 Cohort A1 (no prior anti-PD-1/L1 immunotherapy nor platinum-based combination chemotherapy), and Part 2 Cohort A2 (prior anti-PD-1 monotherapy, platinum-based chemotherapy, or neoadjuvant/adjuvant chemotherapy) (Part 1 Cohort A: n=2 (29%) first-line patients, n=5 (71%) second-line patients; Part 2 Cohort A1: n=22 (100%) first-line patients; Part 2 Cohort A2: n=1 (11%) first-line patients, n=8 (89%) second-line patients). The data was pooled and analyzed by exposure to prior therapy in the locally advanced/metastatic setting (n=25 first-line patients; n=13 second-line patients).

Twenty-five subjects were treatment-naive (first line (1L)) and 13 had prior treatment (second and higher line (2L+)) in the metastatic/locally advanced setting. The treatment-naive subjects had a median age of 64 (48% male), and the prior treatment subjects had a median age of 67 (62% male). Baseline characteristics of the patient population is as follows:

| | First | Second |
|---|---|---|
| | line (1L) | line (2L) |
| | (n = 25) | (n = 13) |
| Median age, years (range) | 64 (46, 82) | 67 (44, 74) |
| Male, n (%) | 12 (48) | 8 (62) |
| ECOG performance score 0/1, n (%) | 12 (48)/13 (52) | 7 (54)/6 (46) |
| Stage III/IV at screening, n (%) | 1 (4)/24 (96) | 0/13 (100) |
| History of brain metastasis, n (%) | 4 (16) | 3 (23) |
| Prior neoadjuvant/adjuvant | 2 (8) | 2 (15) |
| chemotherapy, n (%) | | |
| Prior non-neoadjuvant/adjuvant | 0 | 6 (46) |
| chemotherapy | | |
| Prior anti-PD-1/anti-PD-L1 | 0 | 11 (85) |
| therapy, n (%) | | |
| PD-L1 protein expression, n (%) | | |
| −<1% | 16 (64) | 4 (31) |
| 1%-49% | 5 (20) | 3 (23) |
| −≥50 | 3 (12) | 6 (46) |
| −unknown | 1 (4) | 0 |

### Safety

Treatment-related adverse events (TRAEs) occurred in 36 (95%) subjects, and grade 3-4 in 22 (58%) subjects. The most common Grade 3-4 and TRAEs occurring in ≥5% of all patients were neutropenia/neutrophil count decrease (32%), anemia (21%), and thrombocytopenia/platelet count decrease (16%), ALT increase (5%, Grade 304 ALT increase was observed in the second-line setting only), diarrhea (5%), febrile neutropenia (5%), and nausea (5%). No fatal adverse events occurred. The TRAEs were consistent with sotorasib and platinum doublet-based regimens.

| | Sotorasib + Carboplatin + | |
|---|---|---|
| | Pemetrexed | |
| | First-line | Second-line |
| TRAEs, n (%) | (n = 25) | (n = 13) |
| Any grade | 23 (92) | 13 (100) |
| Grade 1 | 4 (16) | 1 (8) |
| Grade 2 | 7 (28) | 2 (15) |
| Grade 3 | 10 (40) | 8 (62) |
| Grade 4 | 2 (8) | 2 (15) |
| TRAEs leading to discontinuation | 3 (12) | 4 (31) |
| of any treatment | | |
| Discontinuation of sotorasib | 2 (8) | 2 (15) |
| Discontinuation of carboplatin | 1 (4) | 2 (15) |
| Discontinuation of pemetrexed | 3 (12) | 3 (23) |

### Efficacy

In the first line setting (1 L), in which treatment naive subjects were treated with sotorasib in combination with carboplatin and pemetrexed, the ORR (objective response rate) was 65%, with 100% DCR (disease control rate), 95% Confidence Interval (CI) of 83.2-100%. In the second line setting (2L+), ORR was 54%, and 85% DCR, 95% CI 54.6-98.1%.

| | Sotorasib + Carboplatin + | |
|---|---|---|
| | Pemetrexed | |
| Response by Investigator | First-line | Second-line |
| Assessments* | (n = 20) | (n = 13) |
| ORR, n (%) | 13 (65)^{†} | 7 (54) |
| Best overall response, n (%) | | |
| Complete response | 0 | 1 (8) |
| Partial response | 13 (65) | 6 (46) |
| Stable disease | 7 (35) | 4 (31) |
| Progressive disease | 0 | 1 (8) |
| Not evaluable/not done | 0 | 1 (8) |
| DCR | 20 (100) | 11 (85) |
| (95% CI) | (83.2, 100) | (54.6, 98.1) |

*Included were all patients who received ≥1 dose of study drug, had ≥1 measurable lesion at baseline per RECIST v 1.1, and could be followed for ≥7 weeks starting from day 1.
^{†}One additional patient had an unconfirmed PR at Week 6.

The ORR for both 1 L (n=20) and 2L+(n=13) subjects as follows was similar across PD-L1 expression levels. For 1 L subjects (A) with PD-L1 expression levels <1%, the ORR was 62% (n=8/13); (B) with PD-L1 expression 1-49%, the ORR was 75% (n=3/4), and (C) with PD-L1 expression ≥50%, the ORR was 67% (n=2/3). For 2L+subjects (A) with PD-L1 expression levels <1%, the ORR was 50% (n=2/4); (B) with PD-L1 expression 1-49%, the ORR was 67% (n=2/3); and (C) with PD-L1 expression 250%, the ORR was 50% (n=3/6).

With a median follow-up of 3.0 months, preliminary rapid and durable responses were observed. PFS and OS data were immature. Included were all patients in the first- and second-line setting who received ≥1 dose of study drug, had ≥1 measurable lesion at baseline per RECIST v1.1, and could be followed for ≥7 weeks starting from day 1.

### Updated Data Cut Dated Dec. 1, 2023

As of Dec. 1, 2023, 58 patients (median age, 65.5 yrs; 45% male; ECOG status 0/1, 38%/62%) were treated with sotorasib plus carboplatin and pemetrexed; 37 (64%) patients in the first line (1L) and 21 (36%) in the second line plus (2L+) setting. In 2L+, 18/21 (86%) patients had received prior anti-PD-(L)1 therapy. Treatment-related adverse events (TRAEs) occurred in 54 (93%) patients; grade 3-4 TRAEs in 30 (52%) patients and fatal in 1 (2%) (see Table immediately below). In 1L, ORR was 65% (95% CI, 46.5-80.3), DCR was 100%, median DOR was 9.1 months (95% CI, 4.4-12.5), and median PFS was 10.8 months (95% CI, 5.4-NE; median follow-up [f/u], 9.2 months). Median PFS was 11.9 months (95% CI, 5.3-NE) in the PD-L1<1% subgroup (n=19). For 2L+, ORR was 42% (95% CI, 20.3-66.5), DCR was 84%,median DOR was NE, and median PFS was 8.3 months (95% CI, 4.1-NE; median flu, 4.4 mo). OS data remained immature.

TABLE
Key Safety Findings

| n (%) | 1L (n = 37) | 2L+ (n = 21) | Total (N = 58) |
|---|---|---|---|
| TRAEs, any grade | 34 (92) | 20 (95) | 54 (93) |
| Grade 3-4 | 18 (49) | 12 (57) | 30 (52) |
| Fatal* | 0 | 1 (5) | 1 (2) |
| Grade ≥3 TRAE in >10% | | | |
| Neutropenia/Neutrophil | 11 (30) | 6 (29) | 17 (29) |
| count decreased | | | |
| Thrombocytopenia/Platelet | 6 (16) | 3 (14) | 9 (16) |
| count decreased | | | |
| Anemia | 6 (16) | 2 (10) | 8 (14) |
| TRAE leading to | 2 (5)/0/7 | 3 (14)/1 (5)/5 | 5 (9)/1 (2)/12 |
| discontinuation of sotorasib/ | (19)/8 (22) | (24)/6 (29) | (21)/14 (24) |
| carboplatin/pemetrexed/any | | | |
| study drug | | | |

*Fatal TRAE was febrile neutropenia, attributed to carboplatin and pemetrexed.

Sotorasib plus platinum doublet chemotherapy conferred robust and durable responses with a manageable safety profile in CodeBreaK 101, supporting evaluation of this regimen in the ongoing CodeBreaK 202 phase 3 trial in treatment naïve, PD-L1 negative, KRAS G12C-mutated advanced NSCLC (NCT05920356).

### Example 2—a Phase 3 Study of Front Line Platinum Doublet Therapy with Sotorasib Versus Pembrolizumab in PD-L1 Negative KRAS p.G12C Positive Advanced/Metastatic Non-Small Cell Lung Cancer (NSCLC) (CodeBreaK 202)

This is a phase 3, international, multicenter, randomized, open-label study (CodeBreaK 202) to evaluate the efficacy and safety of sotorasib in combination with carboplatin and pemetrexed versus pembrolizumab in combination with carboplatin and pemetrexed in front line setting in subjects with Stage IV or advanced Stage IIIB/C nonsquamous PD-L1 negative and KRAS p.G12C mutation positive non-small cell lung cancer (NSCLC). A Study Evaluating Sotorasib Platinum Doublet Combination Versus Pembrolizumab Platinum Doublet Combination as a Front-Line Therapy in Participants With Stage IV or Advanced Stage IIIB/C Nonsquamous Non-Small Cell Lung Cancers (CodeBreaK 202), https://clinicaltrials.gov/study/NCT05920356, last accessed Feb. 29, 2024.

The study will consist of a pretreatment phase (prescreening period, which is optional for subjects with available molecular data, and a screening period), a treatment phase (4 platinum-containing cycles followed by maintenance therapy), and a posttreatment phase (safety follow-up [SFU] period, and a long-term follow-up [LTFU] period).

The study target population will be subjects with nonsquamous NSCLC who are PD-L1 immunohistochemistry (IHC) negative (Tumor Cell (TC) or Tumor Proportion Score (TPS)<1%) and KRAS p.G12C-mutation positive as determined by the central, or in select cases, local laboratory. The central laboratory will use VENTANA SP263 assay for determination of PD-L1 negativity (TC<1%) and Guardant360® next generation sequencing (NGS) from plasma and Therascreen® KRAS RGQ PCR (polymerase chain reaction) from formalin fixed paraffin embedded (FFPE) tissue for determination of KRAS p.G12C. Positive KRAS p.G12C from either test will be sufficient for enrollment. Enrollment and randomization using local results for PD-L1 IHC and KRAS mutation analysis will be allowed for subjects who need expedited initiation of anticancer therapy due to progressive cancer-related symptoms, provided that results were obtained in qualified laboratory(ies) using qualified assays, and subjects complete and satisfy all other inclusion/exclusion criteria and submit required samples for retrospective molecular pathology confirmation by the central laboratory.

Approximately 750 subjects will be enrolled in the study, which will be conducted at approximately 350 sites globally. Subjects will be randomized in 1:1 ratio to receive either sotorasib in combination with carboplatin and pemetrexed or pembrolizumab in combination with carboplatin and pemetrexed.

Subjects will be stratified by disease stage (Stage IV or advanced Stage IIIB/C, see, AJCC Cancer Staging Manual 8^{th} ed., 2017), brain metastases (yes or no), and region (North America, Europe, or Rest of World). A subject is considered to have brain metastases irrespective of prior treatment of brain lesions.

Subjects in the sotorasib (investigational) treatment group will receive sotorasib 240 mg (or 960 mg) by mouth (PO) once daily (QD) in combination with carboplatin area under the concentration-time curve of 5 mg/mL/min (AUC5) and pemetrexed 500 mg/m², intravenously (IV) every 21 days for 4 cycles, followed by maintenance therapy (cycle 5 and beyond) with sotorasib 240 mg (or 960 mg) PO and pemetrexed 500 mg/m² IV every 21 days. Subjects in the pembrolizumab (control) treatment group will receive pembrolizumab 200 mg in combination with carboplatin AUC5 and pemetrexed 500 mg/m², IV every 21 days for 4 cycles, followed by maintenance treatment (cycle 5 and beyond) with pembrolizumab 200 mg IV (up to 35 cycles) and pemetrexed 500 mg/m² IV every 21 days.

Treatment with sotorasib will continue until Blinded Independent Central Review (BICR) confirmed progressive disease per Response Evaluation Criteria in Solid Tumors version 1.1 (RECIST v1.1), unacceptable toxicity, withdrawal of consent, end of study, or death, whichever occurs first. Pembrolizumab can be administered for up to 2 years. Subjects who discontinue study treatment for reasons other than BICR-confirmed disease progression will continue to have tumor assessments (if clinically feasible) until disease progression is confirmed by BICR.

Study will allow treatment beyond progression only under certain circumstances, as detailed below.

Tumor response will be evaluated by radiological imaging (computed tomography [CT] scan and/or magnetic resonance imaging [MRI]) using RECIST v1.1 criteria.

### Objectives and Endpoints

| | |
|---|---|
| Objectives | Endpoints |

Primary

| | |
|---|---|
| To compare progression- | PFS is defined as the time from |
| free survival (PFS) in | randomization until the first documentation |
| subjects who receive | of radiologic disease progression or death |
| sotorasib with platinum | due to any cause, whichever occurs first. |
| doublet chemotherapy | PFS will be censored at the last evaluable |
| versus subjects who | postbaseline tumor assessment; otherwise, |
| receive pembrolizumab | at randomization. Progression will be based |
| with platinum doublet | on Response Evaluation Criteria in Solid |
| chemotherapy | Tumors (RECIST) v1.1, per Blinded |
| | Independent Central Review (BICR). |

Key Secondary

| | |
|---|---|
| To compare the | Objective response (defined as best overall |
| objective response rate | response of complete response [CR] or |
| (ORR) in subjects who | partial response [PR]) based on RECIST |
| receive sotorasib with | v1.1, per BICR. CR and PR require |
| platinum doublet | confirmatory assessment at least 4 weeks |
| chemotherapy versus | after initial detection of response. |
| subjects who receive | |
| pembrolizumab with | |
| platinum doublet | |
| chemotherapy | |
| To compare overall | OS is defined as the time from |
| survival (OS) in subjects | randomization until death due to any cause. |
| who receive sotorasib | OS will be censored at the last |
| with platinum doublet | contact date known to be alive. |
| chemotherapy versus | |
| subjects who receive | |
| pembrolizumab with | |
| platinum doublet | |
| chemotherapy | |

Secondary

| | |
|---|---|
| To compare progression- | PFS2 is defined as the time from |
| free survival 2 | randomization to progression per investigator |
| (PFS2) in subjects who | after initiation of new anticancer therapy or |
| receive sotorasib with | treatment beyond progression (i.e., second |
| platinum doublet | progression) or death from any cause, |
| chemotherapy versus | whichever occurs first. Subjects alive and for |
| subjects who receive | whom a second disease progression has not |
| pembrolizumab with | been observed will be censored at the last |
| platinum doublet | date known to be alive and without second |
| chemotherapy | progression. |
| To compare patient- | Change from baseline over time to week 12 |
| reported outcomes | in disease-related symptoms of: |
| (PROs) as assessed | Dyspnea as measured by a 4-item |
| by European | dyspnea domain from QLQ-C30 and |
| Organization for | QLQ-LC13 |
| Research and Treatment | Cough as measured by QLQ-LC13 |
| of Cancer (EORTC) | Chest pain as measured by QLQ-LC13 |
| Quality-of-Life | Change from baseline over time to week 12 |
| Questionnaire | in: |
| Core 30 (QLQ-C30) | Physical functioning as measured by |
| and Quality-of-Life | QLQ-C30 |
| Questionnaire Lung | Global health status as measured by |
| Cancer 13 (QLQ-LC13) | QLQ-C30 |
| To compare the effect | Change from baseline over time to week 12 |
| of treatment with | for the remaining subscales for QLQ-LC13 |
| sotorasib with platinum | and QLQ-C30. |
| doublet chemotherapy | Time to deterioration for the subscales for |
| on other treatment-and | QLQ-LC13 and QLQ-C30. |
| disease-related | Summary scores at each assessment and |
| symptoms, and health- | changes from baseline of visual analogue |
| related quality of life | scale (VAS) scores as measured by EuroQol- |
| relative to | 5 Dimension (EQ-5D-5L). |
| pembrolizumab | |
| with platinum | |
| doublet chemotherapy | |
| To compare duration | Duration of response is defined as the time |
| of response, time to | from the first documentation of objective |
| response, and disease | response until the first documentation of |
| control in subjects who | disease progression per BICR or death due |
| receive sotorasib | to any cause, whichever occurs first. Only |
| with platinum doublet | subjects who have achieved objective |
| chemotherapy versus | response per BICR will be evaluated for |
| subjects who receive | duration of response. Censoring rules will |
| pembrolizumab with | follow the primary censoring approach for |
| platinum doublet | PFS. |
| chemotherapy | Time to response is defined as the time from |
| | randomization to first evidence of PR or CR |
| | per BICR. |
| | Disease control is defined as CR plus PR |
| | plus stable disease based on RECIST v1.1 |
| | per BICR after at least 5 weeks. |
| To compare PFS and | PFS based on investigator tumor |
| objective response | assessments per RECIST v1.1. |
| based on investigator | Objective response based on investigator |
| assessment per | tumor assessments per RECIST v1.1. |
| RECIST v1.1 in | |
| subjects who receive | |
| sotorasib with platinum | |
| doublet chemotherapy | |
| versus subjects who | |
| receive pembrolizumab | |
| with platinum doublet | |
| chemotherapy | |
| To compare the safety | Incidence and severity of treatment- |
| and tolerability in | emergent adverse events, changes in |
| subjects who receive | vital signs, and clinical laboratory tests. |
| sotorasib with platinum | |
| doublet chemotherapy | |
| versus subjects who | |
| receive pembrolizumab | |
| with platinum doublet | |
| chemotherapy | |
| To characterize the | PK parameters of sotorasib including, but not |
| pharmacokinetics (PK) | limited to, maximum plasma concentration |
| of sotorasib | (Cₘₐₓ), minimum plasma concentration |
| | (Cₘᵢₙ), area under the plasma |
| | concentration-time |
| | curve (AUC) on day 1, and predose (trough) |
| | concentrations through cycle 4. |

### Estimands for Primary Objective

The hazard ratio (HR) of PFS between subjects who receive sotorasib with platinum doublet chemotherapy versus subjects who receive pembrolizumab with platinum doublet chemotherapy in subjects with Stage IV or advanced Stage IIIB/C nonsquamous NSCLC, negative for PD-L1, and positive for KRAS p.G12C in need of front-line therapy; the intercurrent event is the start of new anticancer therapy before the PFS event, and the primary analysis will censor PFS at the date of last evaluable assessment before or on the start of new anticancer therapy. Sensitivity analysis for PFS including all evaluable assessments after start of new anticancer therapy will be performed.

### Estimands for Key Secondary Objectives:

The difference of proportions of objective response between subjects who receive sotorasib with platinum doublet chemotherapy versus subjects who receive pembrolizumab with platinum doublet chemotherapy in subjects with Stage IV or advanced Stage IIIB/C nonsquamous NSCLC, negative for PD-L1, and positive for KRAS p.G12C in need of front-line therapy. Subjects who start new anticancer therapy before achieving an objective response are considered nonresponders.

The HR of OS between subjects who receive sotorasib with platinum doublet chemotherapy versus subjects who receive pembrolizumab with platinum doublet chemotherapy in subjects with Stage IV or advanced Stage IIIB/C nonsquamous NSCLC, negative for PD-L1, and positive for KRAS p.G12C in need of front-line therapy; the intercurrent event is the start of new anticancer therapy, and the primary analysis will estimate HR of OS regardless of subsequent anticancer therapy.

Change from baseline to week 12 in patient-reported outcome (PRO) endpoints between subjects who receive sotorasib with platinum doublet chemotherapy versus subjects who receive pembrolizumab with platinum doublet chemotherapy in subjects with Stage IV or advanced Stage IIIB/C nonsquamous NSCLC, negative for PD-L1, and positive for KRAS p.G12C in need of front-line therapy. PRO measurements before or on start of new anticancer therapy will be used to estimate treatment effect.

### Summary of Subject Eligibility Criteria

Subjects ages ≥18 years (or legal adult age within country, whichever is older) who have provided informed consent before initiation of any study-specific activities/procedures will be eligible if they satisfy these key inclusion criteria:
histologically or cytologically confirmed diagnosis of nonsquamous histology NSCLC (adenosquamous histology allowed if nonsquamous histology is >50% of the tumor); presence of small cell or large cell neuroendocrine component is an exclusion criterion
Stage IV or advanced Stage IIIB/C NSCLC (not a candidate for definitive multimodality therapy due to the extent of disease)
no history of systemic anticancer therapy in metastatic/incurable settings
must provide tumor tissue sample (or be willing to undergo biopsy) for central molecular analysis
must be tumor tissue negative for PD-L1 expression (TC<1%), tested using SP263 IHC assay
must be plasma circulating tumor DNA (ctDNA) and/or tumor tissue positive for KRAS p.G12C mutation
must have measurable disease as defined by RECIST v1.1 criteria
has Eastern Cooperative Oncology Group (ECOG) Performance Status of 0 or 1

Key exclusion criteria include the following:
mixed histology NSCLC with either small-cell or large-cell neuroendocrine component (any percentage) or predominant squamous cell histology (more than 50% of the available tumor)
tumors known to harbor molecular alterations for which targeted therapy is locally approved (including but not limited to EGFR or ALK alteration) other than KRAS p.G12C
active brain metastases, defined as symptomatic (treated or untreated) brain metastases; subjects with asymptomatic untreated metastases or asymptomatic treated brain metastases are eligible if they meet all prespecified criteria
active autoimmune disease
evidence of active hepatitis B or C (hepatitis panel test performed during screening)
known uncontrolled human immunodeficiency virus (HIV) infection, defined by detectable level of HIV in the blood and/or CD4 level below 400; known controlled HIV allowed

The full list of eligibility criteria is set forth below:

### Complete Inclusion Criteria:

Subjects are eligible to be included in the study only if all of the following criteria apply:
Subject has provided informed consent before initiation of any study-specific activities/procedures.
Subject is age ≥18 years (or legal adult age within country, whichever is older).
Subject has histologically or cytologically confirmed diagnosis of nonsquamous NSCLC.
   If the tumor has mixed histology including a squamous component, the case is suitable for enrollment provided that the squamous component is a minor part of the tumor in the sample (<50% of the tumor present).

Subject has either Stage IV (metastatic) or advanced Stage IIIB or IIIC NSCLC (not a candidate for definitive multimodality therapy due to the extent of disease) as per the AJCC Cancer Staging Manual 8^{th} ed., 2017.

Subject has no history of systemic anticancer therapy in metastatic/non-curable settings. Prior definitive (curative intent) therapy to the early-stage NSCLC will be allowed, if:
Neoadjuvant/adjuvant (to the curative surgery) systemic anticancer therapy, with or without anti-PD-1/L1 therapy, was completed greater than 6 months before the diagnosis of current metastatic disease.
Definitive concurrent chemotherapy with radiation, with or without anti-PD-1/L1 therapy, was completed greater than 6 months before the diagnosis of current metastatic disease.

Subject has provided blood for central analysis of KRAS p.G12C status.

Subject has provided tumor tissue sample in quantity sufficient for central analysis of KRAS p.G12C status.

Subject has provided tumor tissue sample in quantity sufficient for central analysis of PD-L1 status.

Tumor is negative for PD-L1 expression (TC or TPS<1%) assessed either by central laboratory or local laboratory (Clinical Laboratory Improvement Amendments [CLIA]-certified or equivalent per regional standard), using an assay that meets study technical specifications.

Subject must be plasma circulating tumor DNA (ctDNA) or tumor tissue positive for KRAS p.G12C mutation tested by central laboratory testing. In selected cases, local laboratory results can be used for study entry and randomization while central laboratory results are pending.

Subject must have measurable disease as defined by RECIST v1.1 criteria.

Subject has Eastern Cooperative Oncology Group (ECOG) Performance Status of 0 or 1.

Subject's life expectancy is >3 months, in the opinion of the investigator.

Subject can take oral medications and willing to record daily adherence to investigational product.

Subject has adequate hematological laboratory assessments:
Absolute neutrophil count (ANC)≥1.5×10⁹/L.
Hemoglobin ≥9.0 g/dL or >5.6 mmol/L (no blood transfusion 1 week prior).
Platelet count ≥100×10⁹/L.

Subject has adequate renal laboratory assessments and an estimated creatinine clearance (CrCl) 45 mL/min (using Cockcroft-Gault formula, see Cockcroft et al., 1976 and further below).
If the estimated CrCl is below 45 mL/min, then direct measurement of CrCl can be performed, and the subject may enter the study if the actual creatinine clearance (CrCl) is ≥45 mL/min.

Subject has adequate hepatic laboratory assessments, as follows:
AST and ALT<2.5× upper limit of normal (ULN) or ≤5×ULN for subjects with liver metastasis.

$$Total bilirubin \leq 1.5 \times ULN .$$

Subjects with clinically confirmed Gilbert's syndrome with total bilirubin >1.5×ULN are eligible if direct bilirubin within normal limits.

Subject has adequate coagulation laboratory assessments:
International normalized ratio (INR)≥1.5×ULN or within target range if on prophylactic anticoagulation therapy.

Subject has adequate or properly compensated thyroid function as documented by:
Thyroid stimulating hormone (TSH) within normal limits (if TSH is not within normal limits, the subject will be eligible if total triiodothyronine [T3][or free T3], and free thyroxine [T4] are within the normal limits).

Subjects with subclinical hypo- or hyperthyroidism are allowed on the study.

### Complete Exclusion Criteria

Subjects are excluded from the study if any of the following criteria apply:

### Disease Related

Subject has a mixed histology NSCLC with either small-cell or large-cell neuroendocrine cell component (any percentage) or predominant squamous cell histology (more than 50% of the available tumor).
Subjects with tumors known to harbor molecular alterations for which targeted therapy is locally approved (including but not limited to EGFR or ALK alteration) other than KRAS p.G12C.
Subjects with active brain metastases, defined as symptomatic (treated or untreated) brain metastases. Subjects with untreated brain metastases will be allowed into the study, if subject is asymptomatic without systemic steroids, and radiation therapy is not indicated as determined by qualified radiation oncologists.
Subjects with treated brain metastases are eligible if they completed definitive therapy (surgery followed by stereotactic radiation, or stereotactic radiation, or whole brain radiation) and meet all of the following criteria:
   Had at least 7 days of washout period between completion of definitive therapy and cycle 1 day 1., and
   Subject has returned to neurological baseline (apart from residual signs or symptoms related to CNS treatment). Additionally, subjects must be either off corticosteroids, or have decreased the dose to 10 mg daily prednisone (or equivalent) prior to cycle 1 day 1.
Subjects with symptomatic spinal cord compression due to cancer metastases.
Subjects with carcinomatous meningitis (leptomeningeal disease).

### Other Medical Conditions

Subject has history of other malignancy within the past 3 years, with the following exceptions:
Malignancy treated with curative intent and with no known active disease present for ≥2 years before enrollment and felt to be at low risk for recurrence by the treating physician.
Adequately treated non-melanoma skin cancer or lentigo maligna, cervical carcinoma in situ, or breast ductal carcinoma in situ without evidence of disease.
Prostatic intraepithelial neoplasia without evidence of prostate cancer.
Adequately treated urothelial papillary noninvasive carcinoma or carcinoma in situ.
Other malignancies that demonstrated stability over the prior 5 or more years and do not require systemic therapy may be considered upon approval by the medical monitor.

Subject is acutely ill or has an acute surgical condition which may require surgery within first cycle of therapy (as per investigator assessment).

Subject had major surgery (e.g., intrathoracic, abdominal, or vascular surgery that per surgeon assessment requires up to 4 weeks for postoperative healing) within 4 weeks before the first dose of study treatment or the surgical wound has not healed.

Subject has severe active infection requiring antibiotic therapy.

Subject has active autoimmune disease that has required systemic treatment with immunosuppressive/immunomodulating agents (i.e., corticosteroids, inhibitors of tumor necrosis factor alpha, or others) in the past 2 years. The following types of therapies are not considered a form of systemic immunosuppressive therapy and do not exclude subjects from the study:
Replacement therapy (e.g., corticosteroids at physiologic doses) for adrenal insufficiency.
Inhaled steroids.

Subject has interstitial lung disease or a history of pneumonitis that required oral or IV glucocorticoids to assist with management.

Subject has history of solid organ transplantation.

Subject has history of allogenic bone marrow transplantation.

Subject has significant gastrointestinal disorder that results in significant malabsorption requiring IV alimentation, or inability to take oral medication.

Subject has significant cardiovascular disease, such as New York Heart Association cardiac disease (Class III or greater), myocardial infarction within 6 months before randomization, unstable arrhythmias, or unstable angina.

Subject has evidence of active hepatitis B or C (hepatitis panel test performed during screening) defined by positive hepatitis B surface antigen (HepBsAg); or positive hepatitis C antibody and detectable hepatitis C viral RNA:
Subjects with past or resolved HBV infection (defined as the presence of hepatitis B core antibody [anti-HBc]and absence of HBsAg) are eligible.

Subject with known uncontrolled human immunodeficiency virus infection (HIV), defined by detectable level of HIV in the blood and/or CD4 level below 400.
Subjects with known controlled HIV (undetectable by PCR) and compliant with Highly Active Antiretroviral Therapy (HAART) are eligible. These subjects must continue HAART throughout the study treatment as per local standards.

### Prior/Concomitant Therapy

Subject received radiation therapy >30 Gy to the lungs within 6 months of the first dose of study treatment.

Subject completed palliative radiation therapy within 7 days of the first dose of study treatment (for brain radiation).

Subject received prior therapy with sotorasib or other KRAS^{G12C} inhibitor.

Subject received anticancer therapy (chemotherapy, antibody therapy (e.g., anti-VEGF antibody bevacizumab), molecular targeted therapy, hormonal therapy, retinoid therapy, or investigational agent) within past 2 years, except for:
Subject received or continues to receive anticancer hormonal therapy given in adjuvant settings after completely resected early-stage breast cancer with no known active disease for over 2 years, or
Subject with prior neoadjuvant/adjuvant therapy for NSCLC.

Subject used known cytochrome P450 (CYP) 3A4 sensitive substrates or P glycoprotein (P-gp) substrates, both with a narrow therapeutic window, within 14 days before cycle 1 day 1. Exemplary CYP3A sensitive substrates with a narrow therapeutic index include, but are not limited to, alfentanil, fentanyl, cyclosporine, pimozide, dihydroergotamine, quinidine, ergotamine, sirolimus, everolimus, and tacrolimus. Exemplary P-gp substrates with a narrow therapeutic index include, but are not limited to, cyclosporin, sirolimus, digoxin, tacrolimus, everolimus, and vincristine.

Subject used strong inducers of CYP3A4 (including herbal supplements such as St. John's wort) within 14 days before cycle 1 day 1. Examples of CYP3A strong inducers include, but are not limited to, rifampin, mitotane, avasimibe, rifapentine, apalutamide, ivosidenib, phenytoin, carbamazepine, enzalutamide, St John's Wort extract, and lumacaftor.

Subject used proton-pump inhibitors (PPIs) or histamine 2 receptor antagonists (H2RA) within 7 days before cycle 1 day 1.

Subject has received a live-attenuated virus vaccination within 4 weeks of the first dose of study treatment; vaccines that do not contain live virus are permitted.

### Prior/Concurrent Clinical Study Experience

Subject currently receiving treatment in another investigational device or drug study, or less than 4 weeks since ending treatment on another investigational device or drug study(ies). Other investigational procedures while participating in this study are excluded.

### Other Exclusions

Subject has known sensitivity to any of the products or components to be administered during dosing.

Subject likely to not be available to complete all protocol-required study visits or procedures, and/or to comply with all required study procedures (e.g., Clinical Outcome Assessments [COAs]) to the best of the subject and investigator's knowledge.

Subject has history or evidence of any other clinically significant disorder, condition, or disease (with the exception of those outlined above) that, in the opinion of the investigator or medical monitor, if consulted, would pose a risk to subject safety, or interfere with the study evaluation, procedures, or completion.

Female subjects of childbearing potential with a positive pregnancy test assessed at screening and/or cycle 1 day 1 by a highly sensitive urine or serum pregnancy test.

Female subjects of childbearing potential unwilling to use protocol specified method of contraception during treatment with sotorasib, carboplatin, pemetrexed, and pembrolizumab, and for an additional:
7 days after the last dose of sotorasib;
4 months after the last dose of pembrolizumab; and
6 months after the last dose of carboplatin and pemetrexed.

Female subjects planning to become pregnant or donate eggs while on study, during treatment with sotorasib, carboplatin, pemetrexed, and pembrolizumab, and for an additional:
7 days after the last dose of sotorasib;
4 months after the last dose of pembrolizumab; and
6 months after the last dose of carboplatin and pemetrexed.

Female subjects who are breastfeeding or who plan to breastfeed while on study through 7 days after the last dose of sotorasib, carboplatin, and pemetrexed, and 4 months after the last dose of pembrolizumab.

Male subjects with a female partner of childbearing potential who are unwilling to practice sexual abstinence (refrain from heterosexual intercourse) or use contraception during treatment with sotorasib, carboplatin, pemetrexed, and pembrolizumab, and for an additional:
7 days after the last dose of sotorasib and pembrolizumab;
3 months after the last dose of pemetrexed; and
6 months after the last dose of carboplatin.

Male subjects with a pregnant partner who are unwilling to practice abstinence or use a condom during treatment with sotorasib, carboplatin, pemetrexed, and pembrolizumab, and for an additional:
7 days after the last dose of sotorasib and pembrolizumab;
3 months after the last dose of pemetrexed; and
6 months after the last dose of carboplatin.

Male subjects unwilling to abstain from donating sperm during treatment with sotorasib, carboplatin, pemetrexed, and pembrolizumab, and for an additional:
7 days after the last dose of sotorasib and pembrolizumab;
3 months after the last dose of pemetrexed; and
6 months after the last dose of carboplatin.

### Study Intervention

A summary of the dosing and administration of each treatment is detailed below. Investigational product used during this study will be sotorasib and pembrolizumab.

| Study Treatment | Sponsor Investigational Product^{a}: | Non-Sponsor Investigational |
|---|---|---|
| Name | Sotorasib | Product: Pembrolizumab |
| Dosage Formulation | Immediate-release dosage form presented as | 100 mg/4 mL (25 mg/mL) clear to |
| | yellow, oblong, film-coated debossed tablet. | slightly opalescent, colorless to |
| | Excipients used in the tablets include | slightly yellow solution in a single- |
| | microcrystalline cellulose, lactose, | dose vial |
| | croscarmellose sodium, magnesium stearate, | |
| | and Opadry II yellow coating material | |
| | containing polyvinyl alcohol, polyethylene | |
| | glycol, titanium dioxide, talc, and yellow iron | |
| | oxide. | |
| Unit Dose Strength/ | Unit (tablet) dose strength-120 mg | Unit (vial) dose strength-100 mg |
| Dosage Level/ | Dosage level-240 mg (2 tablets) | Dosage level-200 mg |
| Dosage Frequency | Frequency^{b}-once daily (no planned | Frequency^{b}-day 1 of each 21-day |
| | interruptions) in 21-day cycles | cycle (up to 35 cycles) |
| | Dosage level-960 mg (8 tablets) | |
| | Frequency^{b}-once daily (no planned | |
| | interruptions) in 21-day cycles | |
| Route of | Oral | Intravenous |
| Administration | | |
| Dosage Preparation | Not applicable | Dilute pembrolizumab to the final |
| | | concentration between 1 to 10 mg/mL |
| | | in 0.9% Sodium Chloride Injection, |
| | | 5% Dextrose Injection.^{b} |
| Dose | Administer before chemotherapy when given | Administer before chemotherapy |
| Administration | on the same day. Subject should take the | when given on the same day. |
| Instructions | sotorasib dose (all tablets at the same time) at | Administer diluted solution |
| | approximately the same time every day, with | intravenously over 30 minutes |
| | or without food. The sotorasib dose should | through an intravenous line |
| | also not be taken more than 2 hours earlier | containing a sterile, non-pyrogenic, |
| | and 6 hours later than the target time based | low-protein binding, 0.2 to 5 micron |
| | on previous day's dose. If dose is not taken | filter (in-line or add-on). Do not co- |
| | within this window, the dose should be | administer other drugs through the |
| | skipped; take the next dose as prescribed the | same infusion line.^{c} |
| | next day. If vomiting occurs after taking | |
| | sotorasib, do not take an additional dose; take | |
| | the next dose as prescribed the next day. | |

^{a}Sotorasib will be manufactured and packaged by trial sponsor and distributed using sponsor clinical study drug distribution procedures.
^{b}If the initiation of a future cycle is delayed, the ongoing cycle will be continued until the following cycle is started. The start of the following cycle begins with initiation of any study treatment. Once the following cycle is started, the 21-day cycle duration should be reset and maintained. However, if a dose within a cycle is held or missed, the missed dose will not be made up, and day 1 of subsequent cycles should not be adjusted. Further information on the definition and duration of a cycle is provided below.
^{c}Or as per local label/package insert

Non-investigational products (background therapies) used during this study will be carboplatin and pemetrexed, as described below.

| Non-Amgen Non- | | |
|---|---|---|
| Investigational Product | Carboplatin | Pemetrexed |
| Dosage Formulation | Clear, colorless solution in a | Formulated as lyophilized powder or |
| | single-dose vial | solution in a single-dose vial |
| Unit Dose Strength/ | Unit (vial) dose strength-as per local | Unit (vial) dose strength-as per |
| Dosage Level/ | supply | local supply |
| Dosage Frequency | Dosage level-AUC5ª (maximum dose | Dosage level-500 mg/m² |
| | 750 mg) | Frequency^{b}-day 1 of each 21-day |
| | Frequency^{b}-day 1 of each 21-day | cycle |
| | cycle (up to 4 cycles) | |
| Route of Administration | Intravenous | Intravenous |
| Co-administered | Not applicable | Subjects must receive folic acid^{c} and |
| Medications | | vitamin B12^{d} while on pemetrexed, |
| | | see below. |
| Premedication Regimen | Per local standard of care | Per local standard of care^{e} |
| Dosage Preparation | See local label/package insert | See local label/package insert |
| Dose Administration | See local label/package insert | See local label/package insert |
| Instructions | | |

AUC5 = area under the concentration-time curve of 5 mg/ml/min;
GFR = glomerular filtration rate
^{a}The Calvert formula [Total Dose (mg) = (target AUC) × (GFR + 25)] should be used for calculation of carboplatin dose. If an estimated GFR based on measured serum creatinine is used in the Calvert formula, the GFR for the calculation should not exceed 125 mL/min. Based on a target carboplatin AUC of 5, the maximum dose of carboplatin for this study is 750 mg (i.e., AUC 5 mg/mL/min × 150 mL/min).
^{b}If the initiation of a future cycle is delayed, the ongoing cycle will be continued until the following cycle is started. The start of the following cycle begins with initiation of any study treatment. Once the following cycle is started, the 21-day cycle duration should be restored and maintained. However, if a dose within a cycle is held or missed, the missed dose will not be made up, and day 1 of subsequent cycles should not be adjusted. Further information on the definition and duration of a cycle is provided below.
^{c}Low-dose folic acid used on studies ranged from 350 to 1000 μg. The most commonly used dose of oral folic acid in clinical studies was 400 μg. At least 5 daily doses of folic acid must be taken during the 7-day period preceding the first dose of pemetrexed; and dosing should continue during the full course of therapy and for 21 days after the last dose of pemetrexed. Folic acid may be given as per standard of care in accordance with the pemetrexed approved local labeling
^{d}Subjects must receive 1 intramuscular injection of vitamin B12 (dose of 1000 μg) during the week preceding the first dose of pemetrexed and every 9 weeks (± 2 weeks) thereafter. Subsequent vitamin B12 injections may be given the same day as pemetrexed.
^{e}Skin rash due to pemetrexed has been reported more frequently in subjects not pretreated with a corticosteroid. Pretreatment with corticosteroids (dexamethasone or equivalent) reduces the incidence and severity of cutaneous reaction. In clinical studies, dexamethasone 4 mg was given by mouth twice daily the day before, the day of, and the day after pemetrexed administration.

### Other Protocol-Required Therapies

Other Protocol-Required Therapies may include antiemetic therapy before carboplatin administration to prevent nausea/vomiting (NCCN Guidelines, 2022; Multinational Association of Supportive Care in Cancer [MASCC]/ESMO guidelines, Roila et al., 2016), as well as folic acid, vitamin B12, and steroids (dexamethasone or equivalent) alongside pemetrexed administration (see above and refer to pemetrexed approved local labeling).

### Treatment Phase and Definition Thereof

The treatment phase will start on day 1 of cycle 1. Day 1 of cycle 1 will be defined as the first day a subject receives any study-required treatment. Day 1 of the following cycle is a day after last day of the prior cycle. The expected cycle length is about 21 days. The minimum duration of a cycle is 18 days.

Study medications formulation, dosage, frequency, route of administration, accountability, premedication regimen (if any), dosage preparation, and dose administration instructions are described above.

### Dose Adjustments

A maximum of 2 dose reductions related to the toxicities are allowed for sotorasib, carboplatin, or pemetrexed. Subjects who require a third dose reduction of sotorasib, carboplatin, or pemetrexed will have that agent discontinued. Pembrolizumab dose reductions are not permitted. Toxicity needs to resolve to grade ≥1 or baseline before resuming at the subsequent cycle. The rationale for the dose modification and relationship of toxicity to the study agent(s) must be recorded in the source documents. Once the dose is reduced, it may not be re-escalated.

If the study agent(s) was not linked to the toxicity, the investigator may continue this agent(s) at the current dose, assuming safety parameters for this medication met protocol-specified limits (e.g., subjects may have chemotherapy discontinued while continued on sotorasib or pembrolizumab alone; similarly, subjects may discontinue sotorasib or pembrolizumab and continue chemotherapy alone), or hold this study agent(s) until safety parameters for this medication recovered to the acceptable limits (at that time, the noncausative agent may be restarted at the current dose).

Chemotherapy and/or sotorasib may be interrupted for a maximum of 9 weeks; pembrolizumab may be interrupted for a maximum of 12 weeks per instance. If the interruption exceeds the specified period, the medication must be permanently discontinued.

TABLE 4
Dose Levels for Study Medications

| Study Medication | Dose Level 0 | Dose Level −1 | Dose Level −2 | Dose Level −3 |
|---|---|---|---|---|
| Sotorasib^{a} | 960 mg | 480 mg | 240 mg | Discontinue |
| Sotorasib | 240 mg | Dose reductions are not permitted | | |
| Pembrolizumab | 200 mg | Dose reductions are not permitted | | |
| Carboplatin | AUC 5 | AUC 3.75 | AUC 2.5 | Discontinue |
| | Maximum dose | Maximum dose | Maximum dose | |
| | 750 mg | 563 mg | 375 mg | |
| Pemetrexed | 500 mg/m² | 375 mg/m² | 250 mg/m² | Discontinue |

^{a}for the United States, see LUMAKRAS ® US Prescribing Information, Amgen Inc., Thousand Oaks, California, 91320 (revision 01/2023), incorporated herewith in its entirety.

Sotorasib dose reductions are not permitted. Sotorasib will be interrupted or discontinued in the event of a toxicity that, in the opinion of the investigator, is attributed to sotorasib and meets severity that warrants intervention. Guidelines for withholding and permanent discontinuation of sotorasib are listed in Table 5 below. The reason for withholding and permanent discontinuation of sotorasib is to be recorded on each subject's case report form(s) (CRF(s)).

TABLE 5
Sotorasib Dose Modification Guidelines for Sotorasib-related Toxicities

| Toxicity | | |
|---|---|---|
| (Related to Sotorasib) | Severity | Recommended Action |
| Hepatotoxicity^{a,b} | Grade 2 AST or ALT with | Withhold until recovery to grade |
| | symptoms, | ≤1 or to baseline grade |
| | or | Initiate steroids^{c} |
| | Grade 3 to 4 AST or ALT | After recovery, resume dosing at 1 |
| | | lower dose level^{d} |
| | AST or ALT > 3 × ULN with total | Permanently discontinue sotorasib |
| | bilirubin > 2 × ULN in the absence | Initiate steroids^{c} |
| | of alternative causes | |
| Nausea or vomiting despite | Grade 3 to 4 | Withhold until recovery to grade |
| appropriate supportive care | | ≤1 or to baseline grade |
| (including antiemetic therapy) | | After recovery, resume dosing at 1 |
| | | lower dose level |
| Diarrhea despite appropriate | Grade 3 to 4 | Withhold until recovery to grade |
| supportive care (including | | ≤1 or to baseline grade |
| antidiarrheal therapy) | | After recovery, resume dosing at 1 |
| | | lower dose level |
| Suspected interstitial lung | Any grade | If confirmed, permanently |
| disease/pneumonitis | | discontinue sotorasib |
| | | If excluded, resume dosing |
| Any other sotorasib | Grade 3 to 4 | Withhold until recovery to grade |
| | | ≤1 or to baseline grade |
| drug-related toxicity | | After recovery, resume dosing at 1 |
| | | lower dose level |

ALT = alanine aminotransferase;
AST = aspartate aminotransferase;
ULN = upper limit of normal
^{a}For suspected hepatotoxicity refer to the Hepatoxicity Stopping and Rechallenge Rules below.
^{b}For hepatotoxicity that does not meet the severity criteria listed in the table (e.g., grade 2 AST or ALT without associated symptoms), consider steroids, for example: prednisone 1.0 to 2.0 mg/kg/day, dexamethasone equivalent, or methylprednisolone equivalent, followed by a taper over 4 to 6 weeks.
^{c}Initiate steroids, for example: prednisone 1.0 to 2.0 mg/kg/day, dexamethasone equivalent, or methylprednisolone equivalent, followed by a taper over 4 to 6 weeks. The taper may continue while resuming sotorasib (if criteria for resuming sotorasib are met).
^{d}Close monitoring at restart (e.g., repeat liver function tests in 2 to 3 days, then weekly × 3).

### Carboplatin and Pemetrexed

Complete blood cell counts, including platelet counts, should be performed on all subjects receiving carboplatin with pemetrexed, or either of these compounds alone.

Dose adjustments at the start of a subsequent cycle should be based on nadir hematologic counts or maximum non-hematologic toxicity from the preceding cycle of therapy.

Delay initiation of the next cycle of chemotherapy until:
recovery of non-hematologic toxicity to grade 0 to 2, and
ANC is ≥1.5×10⁹/L, and
platelet count is 100×10⁹/L.

Recommended dose modifications for key chemotherapy toxicities are outlined in Table 6 below.

TABLE 6
Dose Modification for Chemotherapy Hematologic Toxicity^{a}

| | | Dose Level From Table 4 | |
|---|---|---|---|
| Platelets and | ANC | Carboplatin | Pemetrexed |
| ≥50 and | <0.5 | −1 | −1 |
| <50 without bleeding and | ANY | −1 | −1 |
| <50 with grade ≥2 | ANY | −2 | −2 |
| bleeding and | | | |
| ANY and | <1.0 + fever | −1 | −1 |
| | (≥38.5° C. | | |
| | or 101° F.) | | |

ANC = absolute neutrophil count;
ANY = any value
^{a}These dose modifications serve as a guide and do not replace investigator judgment and applicable local label recommendations if more stringent.

TABLE 7
Dose Modifications for Chemotherapy Non-hematologic Toxicity^{a}

| | CTCAE | Dose Level From Table 4 | |
|---|---|---|---|
| Event | Grade | Carboplatin | Pemetrexed |
| Nausea or vomiting | 3 or 4 | 0 | 0 |
| Diarrhea | 3 or 4 | 0 | −1 |
| Mucositis | 3 or 4 | 0 | −2 |
| Neurotoxicity | 2 | 0 | 0 |
| | 3 or 4 | −1 | discontinue |
| Transaminase elevation | 3 | −1 | −1 |
| | 4 | discontinue | discontinue |
| Other non-hematologic | 3 or 4 | −1 | −1 |
| toxicity | | | |

CTCAE = Common Terminology Criteria for Adverse Events
^{a}These dose modifications serve as a guide and do not replace investigator judgment and applicable local label recommendations if more stringent.

Recurrent grade 3 or 4 hematological or non-hematologic toxicity after 2 dose reductions should lead to discontinuation of offending chemotherapy agent.

CrCl will be assessed before each cycle using the original weight-based Cockcroft and Gault formula. Chemotherapy (carboplatin and/or pemetrexed) will not be administered if CrCl is less than 45 mL/min. Pemetrexed and/or platinum may be delayed to allow the subject to recover from the toxicity (duration of therapy interruption specified above).

Pemetrexed should be permanently discontinued if it causes interstitial pneumonitis or severe or life-threatening skin toxicity. Please refer to the pemetrexed regional prescribing information (e.g., pemetrexed USPI, SmPC; for the United States, see, e.g., ALIMTA® U.S. Prescribing Information, Lilly USA, LLC, Indianapolis, Indiana 46285 (revision 8/2022), which is herein incorporated by reference in its entirety).

### Pembrolizumab

Pembrolizumab dose reductions are not permitted.

Pembrolizumab may be temporarily withheld as necessary. In general, withhold pembrolizumab for severe (grade 3) immune-mediated adverse reactions. Permanently discontinue pembrolizumab for life-threatening (grade 4) immune-mediated adverse reactions, recurrent severe (grade 3) immune-mediated reactions that require systemic immunosuppressive treatment, or an inability to reduce corticosteroid dose to prednisone 10 mg/day (or equivalent) within 12 weeks of initiating steroids. For guidelines on specific management of immune-mediated adverse reactions and infusion related reactions, refer to the KEYTRUDA® regional prescribing information (e.g., KEYTRUDA® USPI, SmPC; for the United States, see, KEYTRUDA® US Prescribing Information, Merck & Co., Kenilworth, NJ 07033 (revision 3/2023)).

### Hepatoxicity Stopping and Rechallenge Rules

This section details the rules regarding drug-induced liver injury. All study agents in this protocol (sotorasib, carboplatin, pemetrexed, and pembrolizumab) are known to cause elevation of liver enzymes. The investigator should carefully determine causative agents so that proper intervention is applied. Guidelines are provided above for the management of subjects with increases in AST, ALT, or alkaline phosphatase (ALP) related to sotorasib, carboplatin and/or pemetrexed, or pembrolizumab.

Subjects with abnormal hepatic laboratory values (i.e., alkaline phosphatase [ALP], aspartate aminotransferase [AST], alanine aminotransferase [ALT], total bilirubin [TBL]), and/or international normalized ratio (INR) and/or signs/symptoms of hepatitis (as described below) may meet the criteria for withholding or permanent discontinuation of investigational product or other protocol-required therapies, as specified in the Guidance for Industry Drug-Induced Liver Injury: Premarketing Clinical Evaluation, July 2009 (US FDA, 2009).

Criteria for Withholding and/or Permanent Discontinuation of Investigational Product and Other Protocol-required Therapies Due to Potential Hepatotoxicity:

The following stopping and/or withholding rules apply to subjects for whom another cause of their changes in liver biomarkers (TBL, INR, and transaminases) has not been identified. Important alternative causes for elevated AST/ALT and/or TBL values include, but are not limited to:
Hepatobiliary tract disease
Viral hepatitis (e.g., hepatitis A/B/C/D/E, Epstein-Barr Virus, cytomegalovirus, herpes simplex virus, varicella, toxoplasmosis, and parvovirus)
Right-sided heart failure, hypotension, or any cause of hypoxia to the liver causing ischemia
Exposure to hepatotoxic agents/drugs or hepatotoxins, including herbal and dietary supplements, plants, and mushrooms
Heritable disorders causing impaired glucuronidation (e.g., Gilbert's syndrome, Crigler Najjar syndrome) and drugs that inhibit bilirubin glucuronidation (e.g., indinavir, atazanavir)
Alpha-1 antitrypsin deficiency
Alcoholic hepatitis
Autoimmune hepatitis
Wilson's disease and hemochromatosis
Nonalcoholic fatty liver disease, including steatohepatitis
Non-hepatic causes (e.g., rhabdomyolysis, hemolysis)

If investigational product(s) is/are withheld, the subject is to be followed for possible drug-induced liver injury (DILI) according to recommendations in the last section of this appendix.

Rechallenge may be considered if an alternative cause for impaired liver tests (ALT, AST, ALP) and/or elevated TBL, is discovered and the laboratory abnormalities resolve to normal or baseline.

TABLE 8
Conditions for Withholding and/or Permanent Discontinuation of Investigational Product and
Other Protocol-required Therapies Due to Potential Hepatotoxicity

| Analyte | Temporary Withholding | Permanent Discontinuation |
|---|---|---|
| TBL | >3 × ULN | >2 × ULN |
| | at any time | OR |
| INR | — | >1.5 × (for subjects not on anticoagulation |
| | | therapy) |
| | OR | AND |
| AST/ALT | >8 × ULN at any time | >3 × ULN (when baseline was < ULN), in |
| | >5 × ULN but <8 × ULN for ≥2 weeks | the presence of no important alternative |
| | >5 × ULN but <8 × ULN and unable to | causes for elevated AST/ALT and/or TBL |
| | adhere to enhanced monitoring schedule | values |
| | >3 × ULN with clinical signs or symptoms that | |
| | are consistent with hepatitis (such as right | |
| | upper quadrant pain/tenderness, fever, | |
| | nausea, vomiting, and jaundice) | |
| | OR | |
| ALP | >8 × ULN at any time | |

ALP = alkaline phosphatase;
ALT = alanine aminotransferase;
AST = aspartate aminotransferase;
INR = international normalized ratio;
TBL = total bilirubin;
ULN = upper limit of normal

Criteria for Rechallenge of Investigational Product and Other Protocol required Therapies After Potential Hepatotoxicity:

The decision to rechallenge the subject is to be discussed and agreed upon unanimously by the subject, investigator, and Medical Monitor. If signs or symptoms recur with rechallenge, then investigational product and other protocol-required therapies, as appropriate, are to be permanently discontinued. Subjects who clearly meet the criteria for permanent discontinuation (as described in Table 8) are never to be rechallenged.

Response Evaluation Criteria in Solid Tumors Version 1.1 (RECIST v1.1) (Eisenhauer et al., 2009; Schwartz et al., 2016; Therasse et al., 2000).

### Definitions

### Measurable Disease

The presence of at least 1 measurable lesion. If the measurable disease is restricted to a solitary lesion, its neoplastic nature should be confirmed by cytology/histology.

### Measurable Lesions

### Measurable Non-Nodal Tumor Lesions

Non-nodal lesions with clear borders that can be accurately measured in at least 1 dimension with longest diameter ≥10 mm in computed tomography (CT)/magnetic resonance imaging (MRI) scan with slice thickness no greater than 5 mm. When slice thickness is greater than 5 mm, the minimum size of measurable lesion should be twice the slice thickness.

### Nodal Lesions

Lymph nodes are to be considered measurable if ≥15 mm in short axis when assessed by CT/MRI (scan slice thickness recommended to be no greater than 5 mm). At baseline and in follow-up, only the short axis will be measured and followed (Schwartz et al., 2009).

### Cystic Lesions

Cystic lesions thought to represent cystic metastases can be considered as measurable lesions, if they meet the definition of measurability described above for non-nodal lesions.

Bone Lesions with Identifiable Soft Tissue Components

Bone lesions with identifiable soft tissue components, that can be evaluated by cross sectional imaging techniques such as CT or MRI can be considered as measurable lesions if the soft tissue component meets the definition of measurability described above for non-nodal lesions.

### Clinically Measured Lesions

Visible or palpable lesions can be considered measurable if ≥10 mm in longest diameter for non-nodal or ≥15 mm in shortest diameter for lymph nodes. Lesions should be measured radiologically if more accurate, if not then measured by calipers.

### Irradiated Lesions

Tumor lesions situated in a previously irradiated area, or in an area subjected to other locoregional therapy, are not measurable unless there has been demonstrated progression that is measurable in the lesion before enrollment.

### Non-Measurable Lesions

All other lesions, including small lesions (longest diameter <10 mm or pathological lymph nodes with ≥10 mm to <15 mm short axis with CT scan slice thickness no greater than 5 mm) are considered non measurable. (When slice thickness is greater than 5 mm, the minimum size of measurable lesion should be twice the slice thickness).

Other examples of lesions usually considered to be non-measurable include:

Lesions with prior local treatment: tumor lesions situated in a previously irradiated area, or an area subject to other locoregional therapy, should not be considered measurable unless there has been demonstrated progression in the lesion.

Categorically, clusters of small lesions, bone lesions without a soft tissue component, inflammatory breast disease, ascites, pleural/pericardial effusions, lymphangitis cutis/pulmonitis, and leptomeningeal disease are non-measurable.

### Methods of Measurement

All measurements should be taken and recorded in metric notation, using a ruler or calipers. The same method of assessment and the same technique should be used to characterize each identified and reported lesion at baseline and throughout the trial. Imaging-based evaluation is preferred to evaluation by clinical examination unless the lesion(s) being followed cannot be imaged but are assessable by clinical exam. Clinical lesions will be assessed using calipers (e.g., skin nodules). In the case of skin lesions, documentation by color photography, including a ruler to estimate the size of the lesion, is recommended.

### CT/MRI

Contrast-enhanced CT or MRI should be used to assess all lesions. Optimal visualization and measurement of metastasis in solid tumors requires consistent administration (dose and rate) of intravenous (IV) contrast as well as timing of scanning. CT and MRI should be performed with ≤5 mm thick contiguous slices. The longest diameter of selected lesions should be measured in the plane in which the images were acquired. Ideally, the same scanner or at least type of scanner should be used, and the image acquisition protocol should be followed as closely as possible to prior scans.

### PET-CT

At present, the low dose or attenuation correction CT portion of a combined PET-CT is not always of optimal diagnostic CT quality for use with RECIST measurements. However, if the site can document that the CT performed as part of a PET-CT is of identical diagnostic quality to a diagnostic CT (with IV and oral contrast), then the CT portion of the PET-CT can be used for RECIST measurements and can be used interchangeably with conventional CT in accurately measuring cancer lesions over time. Note, however, that the PET portion of the CT introduces additional data which may bias an investigator if it is not routinely or serially performed.

### Ultrasound

Ultrasound is not useful in assessment of lesion size and should not be used as a method of measurement. Ultrasound examinations cannot be reproduced in their entirety for independent review at a later date, and because they are operator dependent, it cannot be guaranteed that the same technique and measurements will be taken from one assessment to the next. If new lesions are identified by ultrasound in the course of the study, confirmation by CT or MRI is advised. If there is concern about radiation exposure at CT, MRI may be used instead of CT in selected instances.

### Endoscopy, Laparoscopy

The utilization of these techniques for objective tumor evaluation is not advised. However, such techniques may be useful to confirm complete pathological response when biopsies are obtained or to determine relapse in trials where recurrence following complete response (CR) or surgical resection is an endpoint.

### Tumor Markers

Tumor markers alone cannot be used to assess response. If markers are initially above the upper normal limit, they must normalize in a subject with a radiological CR for a subject to be considered a CR.

### Cytology, Histology

These techniques can be used to differentiate between PR and CR in rare cases if required by protocol (for example, residual lesions in tumor types such as germ cell tumors, where known residual benign tumors can remain).

When effusions are known to be a potential adverse effect of treatment (e.g., with certain taxane compounds or angiogenesis inhibitors), the cytological confirmation of the neoplastic origin of any effusion that appears or worsens during treatment can be considered if the measurable tumor has met criteria for response or stable disease to differentiate between response (or stable disease) and progressive disease.

### FDG-PET

While fluorodeoxyglucose-positron emission tomography (FDG-PET) response assessments need additional study, it is sometimes reasonable to incorporate the use of FDG-PET scanning to complement CT scanning in assessment of progression (particularly possible ‘new’ disease). New lesions on the basis of FDG-PET imaging can be identified according to the following algorithm:

Negative FDG-PET at baseline, with a positive FDG-PET at follow-up is a sign of progressive disease based on a new lesion.

No FDG-PET at baseline and a positive FDG-PET at follow-up: If the positive FDG PET at follow-up corresponds to a new site of disease confirmed by CT, this is progressive disease. If the positive FDG-PET at follow-up is not confirmed as a new site of disease on CT, additional follow-up CT scans are needed to determine if there is truly progression occurring at that site (if so, the date of progressive disease will be the date of the initial abnormal FDG PET scan). If the positive FDG-PET at follow-up corresponds to a preexisting site of disease on CT that is not progressing on the basis of the anatomic images, this is not progressive disease.

FDG-PET may be used to upgrade a response to a CR in a manner similar to a biopsy in cases where a residual radiographic abnormality is thought to represent fibrosis or scarring.

Note: A ‘positive’ FDG-PET scan lesion means one which is FDG avid with an uptake greater than twice that of the surrounding tissue on the attenuation corrected image.

### Lesion Evaluation

Baseline Documentation of “Target” and “Non-target” Lesions

### Target Lesions

All measurable lesions up to a maximum of 2 lesions per organ and 5 lesions in total, representative of all involved organs should be identified as target lesions and recorded and measured at baseline.

Target lesions should be selected on the basis of their size (lesions with the longest diameter) and suitability for accurate repeated measurements. All other measurable lesions will be followed as non-target lesions.

Lymph nodes are considered 1 organ, thus a maximum of 2 measurable lymph nodes may be identified as target lesions.

A sum of the diameters (longest for non-nodal lesions, short axis for nodal lesions) for all target lesions will be calculated and reported as the baseline sum of diameters. The baseline sum of diameters will be used as reference by which to characterize objective tumor response.

### Non-Target Lesions

All other lesions (or sites of disease) including pathological lymph nodes should be identified as non-target lesions and should be recorded at baseline. These lesions should be followed as “present,” “absent,” “unequivocal progression,” or “not evaluable” (NE) throughout the study. In addition, it is possible to record multiple non-target lesions involving the same organ as a single item on the case report form (e.g., “multiple enlarged pelvic lymph nodes” or “multiple liver metastases”).

TABLE 9
Response Criteria. Evaluation of Target Lesions

| | |
|---|---|
| Complete Response (CR) | Disappearance of all target non-nodal lesions. Any target lymph |
| | node must have reduction in short axis to <10 mm, NOT total |
| | disappearance. |
| Partial Response (PR) | At least a 30% decrease in the sum of the diameters of target |
| | lesions, taking as reference the baseline sum of diameters. |
| Progressive Disease (PD) | At least a 20% increase in the sum of the diameters of target |
| | lesions, taking as reference the smallest sum on study (this |
| | includes the baseline sum if that is the smallest on study). In |
| | addition to the relative increase of 20%, the sum must also |
| | demonstrate an absolute increase of at least 5 mm. If a subject is |
| | missing lesion data at a disease assessment and yet progressive |
| | disease criteria is met despite the missing data, the subject will be |
| | classified as PD. |
| Stable Disease (SD) | Neither sufficient shrinkage to qualify for PR nor sufficient increase |
| | to qualify for PD. |
| Not Evaluable (NE) | When inadequate or no imaging/measurement is done at a |
| | particular time point, the subject's response is NE at that time |
| | point. |

TABLE 10
Response Criteria. Evaluation of Non-target Lesions

| | |
|---|---|
| Complete Response (CR) | Disappearance of all non-nodal non-target lesions and |
| | normalization of tumor marker level. All non-target lymph nodes |
| | must be non-pathological in size (<10 mm short axis). |
| Non-CR/Non-PD | Persistence of one or more non-target lesions(s) and/or |
| | maintenance of tumor marker level above the normal limits. |
| Progressive Disease (PD) | Unequivocal progression of existing non-target lesions.ª If a |
| | subject is missing lesion data at a disease assessment and yet |
| | unequivocal progression is met despite the missing data, the |
| | subject will be classified as PD. |
| Not Evaluable (NE) | When inadequate or no imaging is done at a particular time point, |
| | the subject's response is NE at that time point. |

^{a}To achieve “unequivocal progression” on the basis of the non-target disease, there must be an overall level of substantial worsening in non-target disease such that, even in presence of SD or PR in target disease, the overall tumor burden hasincreased sufficiently to merit discontinuation of therapy. A modest “increase” in the size of 1 or more non-target lesions is usually not sufficient to qualify for unequivocal progression status.

### Evaluation of Best Overall Response

The subject's best response assignment will depend on the findings of both target and non target disease and will also take into consideration the appearance of new lesions. BOR will be based on all postbaseline disease assessments that occur before the initiation of subsequent anticancer treatment. At least 5 weeks from cycle 1 day 1 must elapse without radiological disease progression to meet the minimum criteria for SD duration in order to assign a BOR of SD. In general, subjects not classifiable under the RECIST v1.1 response categories due to inadequate data or early death will be classified as NE for BOR but will be counted in the denominator of all response rate calculations.

TABLE 11
Time Point Overall Response

| | | | |
|---|---|---|---|
| Target Lesions | Non-target Lesions | New Lesions | Overall Response |

Subjects With Target (± Non-target) Disease

| | | | |
|---|---|---|---|
| CR | CR or NA | No | CR |
| CR | Non-CR/non-PD or NE | No | PR |
| PR | CR or Non-CR/Non-PD or NE | No | PR |
| | or NA | | |
| SD | CR or Non-CR/Non-PD or NE | No | SD |
| | or NA | | |
| PD | Any | Any | PD |
| Any | PD | Any | PD |
| Any | Any | Yes | PD |
| NE | CR or Non-CR/Non-PD or NE | No | NE |
| | or NA | | |

Subjects With Non-target Disease Only

| | | | |
|---|---|---|---|
| NA | CR | No | CR |
| NA | Non-CR/Non-PD | No | Non-CR/Non-PD^{a} |
| NA | CR or Non-CR/Non-PD | NE | Non-CR/Non-PD^{a} |
| NA | PD | Any | PD |
| NA | Any | Yes | PD |
| NA | NE | No | NE |

^{a}To achieve “unequivocal progression” on the basis of the non-target disease, there must be an overall level of substantial worsening in non-target disease such that, even in presence of SD or PR in target disease, the overall tumor burden has increased sufficiently to merit discontinuation of therapy. A modest “increase” in the size of 1 or more non-target lesions is usually not sufficient to qualify for unequivocal progression status.

TABLE 12
Best Overall Response when Confirmation of Complete Response (CR)
and Partial Response (PR) Required

| Overall Response | Overall Response | |
|---|---|---|
| First Time Point | Second Time Point | Best Overall Response |
| CR | CR | CR |
| CR | PR | SD, PD or PR^{a} |
| CR | SD | SD provided minimum criteria for SD |
| | | duration met, otherwise, PD |
| CR | PD | SD provided minimum criteria for SD |
| | | duration met, otherwise, PD |
| CR | NE | SD provided minimum criteria for SD |
| | | duration met, otherwise, NE |
| PR | CR | PR |
| PR | PR | PR |
| PR | SD | SD |
| PR | PD | SD provided minimum criteria for SD |
| | | duration met, otherwise, PD |
| PR | NE | SD provided minimum criteria for SD |
| | | duration met, otherwise, NE |
| NE | NE | NE |

CR = complete response;
PD = progressive disease;
PR = partial response;
NE = not evaluable;
SD = stable disease
^{a}If a CR is truly met at first time point, then any disease at a subsequent time point (see Confirmation section below for timing), even disease meeting PR criteria relative to baseline, makes the disease PD at that point (since disease must have reappeared after CR). Best response would depend on whether minimum duration for SD was met. However, sometimes “CR” may be claimed when subsequent scans suggest small lesions were likely still present and in fact that subject had PR, not CR at the first time point. Under these circumstances, the original CR should be changed to PR and the best response is PR

### Special Notes of Response Assessment

Target lesions that become “too small to measure”—While on study, all lesions (nodal and non-nodal) recorded at baseline should have their measurements recorded at each subsequent evaluation, even when very small (e.g., 2 mm). However, sometimes lesions or lymph nodes which are recorded as target lesions at baseline become so faint on CT scan that the radiologist may not feel comfortable assigning an exact measure and may report them as being ‘too small to measure’. When this occurs, it is important that a value be recorded on the case report form. If it is the opinion of the radiologist that the non-lymph node lesion has likely disappeared, the measurement should be recorded as 0 mm. If the lesion is believed to be present and is faintly seen but too small to measure, a default value of 5 mm should be assigned. (Note: It is less likely that this rule will be used for lymph nodes since they usually have a definable size when normal and are frequently surrounded by fat such as in the retroperitoneum; however, if a lymph node is believed to be present and is faintly seen but too small to measure, a default value of 5 mm should be assigned in this circumstance as well.) This default value is derived from the 5 mm CT slice thickness (but should not be changed with varying CT slice thickness). The measurement of these lesions is potentially non-reproducible, therefore providing this default value will prevent false responses or progressions based upon measurement error. To reiterate, however, if the radiologist is able to provide an accurate measure, that should be recorded, even if it is below 5 mm.

New lesions—The term “new lesion” always refers to the presence of a new finding that is definitely tumor. If a new lesion is identified via a modality other than CT or MRI, CT or MRI confirmation is recommended unless the new lesion is deemed unequivocally tumor. New findings that are not definitively tumor but may be benign (infection, inflammation, etc) are not selected as new lesions, until that time when the review is certain they represent tumor.

If a new lesion is equivocal, for example because of its small size, continued therapy and follow-up evaluation will clarify if it represents truly new disease. If additional imaging confirms there is definitely a new lesion, then progression should be declared using the date of the initial scan.

A lesion identified on a follow-up study in an anatomical location that was not scanned at baseline is considered a new lesion and will indicate disease progression, regardless of any response that may be seen in target or non-target lesions present from baseline.

### Any Locoregional Therapy not Allowed Per Protocol

Any subject receiving locoregional therapy not allowed in the protocol while on study that directly affects 1 or more of the target lesions selected at baseline will be considered to be non-evaluable at all disease assessments that occur on or after the date of locoregional therapy with the exception of disease progression. However, if a lesion was completely resected where pathology was benign, the subject will still be evaluable for response with 0 dimension reported.

If locoregional therapy was performed on a non-target lesion, that lesion will always be assessed as present unless pathology was benign.

Lesions that split or coalesce on treatment—When non-nodal lesions “fragment,” the longest diameters of the fragmented portions should be added together to calculate the target lesion sum and identified as a fragment of the original lesion. Similarly, as lesions coalesce, a plane between them may be maintained that would aid in obtaining maximal diameter measurements of each individual lesion. If the lesions have truly coalesced such that they are no longer separable, the vector of the longest diameter in this instance should be the maximal longest diameter for the “coalesced lesion.”

“Symptomatic deterioration” alone does not qualify as objective progression. If objective progression was not previously documented, then every effort should be made to document objective progression even after discontinuation of treatment.

In some circumstances it may be difficult to distinguish residual disease from scar or normal tissue. When the evaluation of CR depends on this determination, it is recommended that the residual lesion be further investigated by fine needle aspirate/biopsy or FDG-PET, to confirm the CR status.

If a lesion disappears and reappears at a subsequent time point it should continue to be measured. However, the subject's response at the point in time when the lesion reappears will depend upon the status of his/her other lesions. For example, if the subject's tumor had reached a CR status and the lesion reappeared, then the subject would be considered PD at the time of reappearance. In contrast, if the tumor status was a PR or SD and 1 lesion which had disappeared then reappears, its maximal diameter should be added to the sum of the remaining lesions for a calculated response: in other words, the reappearance of an apparently “disappeared” single lesion amongst many which remain is not in itself enough to qualify for PD: that requires the sum of all lesions to meet the PD criteria.

### Confirmation Measurement/Duration of Response

Confirmation of CR and PR is required and must occur no fewer than 4 weeks after initial documentation of CR or PR. If CR is pending confirmation and is designated at an assessment followed by 1 or more NE assessments, and/or PR assessments such that the Target Lesion Response is CR and the Non-Target Lesion Response is NE, CR may be confirmed thereafter if Non-Target Lesion Response returns to CR. Similarly, if a PR is pending confirmation and is designated at an assessment followed by 1 or more NE and/or SD assessments, PR may be confirmed thereafter. Subsequent Target Lesion Responses following a CR are limited to CR, PD or NE; PD for target lymph nodes is met only if any lymph node target lesion reaches a short axis measurement of ≥15 mm.

### Response Assessment in Neuro-Oncology Brain Metastases (RANO-BM) Criteria

This study will include an exploratory analysis of intracranial PFS according to the Response Assessment in Neuro-oncology Brain Metastases (RANO-BM) (Lin et al., 2015) criteria per BICR. RANO-BM is an extension of the Response Evaluation Criteria in Solid Tumors (RECIST) 1.1 (Eisenhauer et al., 2009) and the Response Assessment in Neuro oncology (RANO) (Wen et al., 2010) response assessment criteria for high-grade gliomas.

### Definitions

Measurable central nervous system (CNS) disease is defined as a contrast enhancing CNS lesion that can be accurately measured in at least one dimension, with a minimum size of 10 mm, and is visible on two or more axial slices that are preferably 5 mm or less apart with 0 mm skip (and ideally ≤1.5 mm apart with 0 mm skip).
The diameter perpendicular to the longest diameter in the plane of measurement should be at least 5 mm for the CNS lesion to be considered measurable.
When more than one measurable CNS lesion is present at baseline, all CNS lesions (up to a maximum of 5) should be recorded and measured at baseline.
If CNS lesions that are >5 mm but <10 mm are to be considered as measurable disease, CNS lesions should be measured with magnetic resonance imaging (MRI) imaging with 1.5 mm slice thickness or less. Any CNS lesions <10 mm in the longest diameter should be regarded as unchanged from baseline unless there is a minimum 3 mm change in the measured longest diameter.

Non-measurable CNS lesions include all other CNS lesions, including lesions with longest dimension <10 mm, lesions with borders that cannot be reproducibly measured, dural metastases, bony skull metastases, cystic-only lesions disease.

### Methods of Measurement

Measurement of lesions: The longest diameter of selected lesions should be measured in the place in which the images were acquired.

Methods of Assessment: The same method of assessment and the same technique should be used to characterize each identified and reported lesion at baseline and throughout the trial.

MRI: Gadolinium-enhanced MRI should be used to assess all CNS lesions. Computed tomography (CT) with and without contrast may be performed if MRI is contraindicated or inaccessible.

### Response Assessment for CNS Target Lesions

| | |
|---|---|
| Complete Response (CR) | Disappearance of all CNS target lesions sustained for at least 4 |
| | weeks; with no new lesions, no use of corticosteroids, and subject |
| | is stable or improved clinically. |
| Partial Response (PR) | At least a 30% decrease in the sum longest diameter of CNS target |
| | lesions, taking as reference the baseline sum longest diameter |
| | sustained for at least 4 weeks; no new lesions; stable to decreased |
| | corticosteroid dose; stable or improved clinically. |
| Stable Disease (SD) | Neither sufficient shrinkage to qualify for PR nor sufficient increase |
| | to qualify for progressive disease, taking as reference the smallest |
| | sum longest diameter while on study. |
| Progressive Disease (PD) | At least a 20% increase in the sum longest diameter of CNS target |
| | lesions, taking as reference the smallest sum on study (this |
| | includes the baseline sum if that is the smallest on study). In |
| | addition to the relative increase of 20%, at least one lesion must |
| | increase by an absolute value of 5 mm or more to be considered |
| | progression. |

For lesions that coalesce, a plane between them may be maintained that would aid in obtaining the maximum longest diameter of each individual lesion. If the lesions have coalesced and are no longer separable, the vector of the longest diameter in this instance should be the maximum longest diameter in this instance should be the maximum longest diameter for the coalesced lesion.

New CNS lesions, which were not present on prior scans, should be unequivocal and not due to technical or slice variation.

Non-target lesions should be assessed at least qualitatively with each radiographic assessment.

### Response Assessment for Non-Target Lesions

| | |
|---|---|
| Complete Response (CR) | Disappearance of all enhancing CNS non-target |
| | lesions, no new CNS lesions. |
| Non-complete response or non-progressive | Persistence of one of more non-target CNS |
| disease | lesion(s). |
| Progressive Disease (PD) | Any of the following: unequivocal progression of |
| | existing enhancing non-target CNS lesions, new |
| | lesion(s), or unequivocal progression of existing |
| | tumor-related non-enhancing (T2/FLAIR) CNS |
| | lesions. |

CNS and non-CNS compartments will be assessed separately as follows:

| CNS | Non-CNS | |
|---|---|---|
| (RANO-BM) | (RECIST 1.1) | Response |
| CR, PR, or SD | CR, PR, or SD | CNS and non-CNS CR, PR, or |
| | | SD, respectively |
| CR, PR, or SD | PD | CNS CR, PR, or SD respectively; |
| | | non-CNS PD |
| PD | CR, PR, or SD | CNS PD; non-CNS CR, PR, or |
| | | SD, respectively |
| PD | PD | CNS and non-CNS PD |

For assessment of overall progression free survival, if progression occurs in either or both compartments, the criterial for progression-free survival will have been met. Subjects who develop isolated CNS progression may be eligible to remain on study after local therapy (e.g., whole-brain radiotherapy, stereotactic radiosurgery, or surgery).

### Eastern Cooperative Oncology Group (ECOG) Performance Status

ECOG Performance Status Scale

| Grade | Descriptions |
|---|---|
| 0 | Fully active, able to carry on all pre-disease performance without restriction. |
| 1 | Restricted in physically strenuous activity, but ambulatory and able to carry out work of a |
| | light or sedentary nature (e.g., light housework, office work). |
| 2 | Ambulatory and capable of all self-care, but unable to carry out any work activities. Up and |
| | about more than 50% of waking hours. |
| 3 | Capable of only limited self-care, confined to bed or chair more than 50% of waking hours. |
| 4 | Completely disabled. Cannot carry on any self-care. Totally confined to bed or chair. |
| 5 | Dead. |

ECOG = Eastern Cooperative Oncology Group Source: Oken et al., 1982.

### Calculate Creatinine Clearance

Creatinine clearance is for the purposes of this protocol is calculated as follows:
Original, Weight-Based Cockcroft and Gault Formula (see, Cockcroft et al., 1976)
For Men
   For serum creatinine concentration in mg/dL:

$$\begin{matrix}CrCl \\ ( mL / min )\end{matrix} = \frac{( 140 - {age}^{a} ) \times ( {wt}^{b} ) \times 1.}{72 \times serum creatinine ( mg / dL )}$$

For serum creatinine concentration in μmol/L:

$$\begin{matrix}CrCl \\ ( mL / min )\end{matrix} = \frac{( 140 - {age}^{a} ) \times ( {wt}^{b} ) \times 1.}{0.81 \times serum creatinine ( μ mol / dL )}$$

$${a}^{} Age in years .$$

$${b}^{} Weight ( wt ) in kilograms .$$

$$Source : Cockcroft and Gault 1976.$$

For Women
   For serum creatinine concentration in mg/dL:

$$\begin{matrix}CrCl \\ ( mL / min )\end{matrix} = \frac{( 140 - {age}^{a} ) \times ( {wt}^{b} ) \times 0.85}{72 \times serum creatinine ( mg / dL )}$$

For serum creatinine concentration in μmol/L:

$$\begin{matrix}CrCl \\ ( mL / min )\end{matrix} = \frac{( 140 - {age}^{a} ) \times ( {wt}^{b} ) \times 0.85}{0.81 \times serum creatinine ( μ mol / dL )}$$

$${a}^{} Age in years .$$

$${b}^{} Weight ( wt ) in kilograms .$$

$$Source : Cockcroft and Gault 1976.$$

### REFERENCES

Addeo, et al. Cancer Treat. Rev. 2021; 96(102179): 1-10.
Ahn, et al. J. Thorac. Oncol. 2016; 11(4): S115.
Amin, et al., eds. AJCC Cancer Staging Manual. 8th ed. New York: Springer; 2017.
ALIMTA® U.S. Prescribing Information, Lilly USA, LLC, Indianapolis, Indiana 46285 (revision 08/2022).
ALIMTA® U.S. Prescribing Information, Lilly USA, LLC, Indianapolis, Indiana 46285 (as approved Aug. 19, 2004), available at https://www.accessdata.fda.gov/drugsatfda_docs/label/2004/021677lbl.pdf, last accessed Mar. 30, 2023.
Besse, et al. Annals of Oncology 2014; 25:1475-1484.
Blank, et al. Cancer Immunol. Immunother. 2007; 56(5):739-745.
Butte, et al. Immunity 2007; 27(1):111-122.
Calvert, et al. J. Clin. Oncol. 1989; 7:1748-1756.
Canon, et al. Nature 2019; 575(7781), 217.
CARBOplatin Injection, U.S. Prescribing Information, Fresenius KABI, Lake Zurich, Illinois, 60047 (revision 5/2021).
Cerami, et al. Cancer Discov. 2012; 2(5):401-404.
Cockcroft, Gault, Nephron. 1976; 16:31-41.
CodeBreaK 101, ClinicalTrials.gov identifier: NCT04185883, https://clinicaltrials.gov/ct2/show/NCT04185883, last accessed Mar. 27, 2023.
Dong, et al. Nat. Med. 1999; 5(12): 1365-1369.
Eisenhauer, et al., Eur. J. Cancer 2009 45:228-247.
ESMO Clinical Practice Guidelines—metastatic NSCLC. Updated September 2020, originally published Ann Oncol. 2018; 29 (suppl 4):iv192-iv237 https://www.esmo.org/guidelines/lung-and-chest-tumours/clinical-practice-living-guidelines-metastatic-non-small-cell-lung-cancer.
Gadgeel, et al. Ann. Oncol. 2019; 30(11). Late-breaking abstract 5.
Gadgeel, et al. J. Clin. Oncol. 2020; 38(14):1505-1517.
Gandhi, et al. N. Engl. J. Med. 2018; 378(22):2078-2092.
Gao, et al. Sci. Signal. 2013; 6(269):p11, pages 1-19.
Gibbons, et al. J. Thorac. Oncol. 2016; 11(4):S79.
GLOBOCAN 2020—Lung. Available at http://gco.iarc.fr/today/home. Last accessed March 2022.
Gogishvili, et al. Nat. Med., 2022; 28:2374-2380.
Hendriks, et al. Ann. Oncol. 2023a. doi: https://doi.org/10.1016/j.annonc.2022.12.009.
Hendriks, et al. Ann. Oncol. 2023b. doi: https://doi.org/10.1016/j.annonc.2022.12.013.
Herbst, et al. Ann. Oncol. 2019; 30(11). Late-breaking abstract 4.
Hong, et al. N. Engl. J. Med. 2020; 383:1207-1217.
Janes et al. Cell. 2018; 172(3):578-589.
Jeanson, et al. J. Thorac. Oncol. 2019; 14(6):1095-1101.
Keir, et al. Annu. Rev. Immunol. 2008; 26:677-704.
KEYTRUDA® US Prescribing Information, Merck & Co., Kenilworth, NJ 07033 (revision 3/2023).
Kris, et al. J. Clin. Oncol. 2017; 35(25):2960-2974.
Langer, et al. Lancet Oncol. 2016; 17(11):1497-1508.
Lanman, et al. J. Med. Chem. 2020; 63(1):52-65.
Lin, et al. Lancet Oncol. 2015; 16(6):e270-e278.
LUMAKRAS® US Prescribing Information, Amgen Inc., Thousand Oaks, California, 91320 (revision 01/2023).
Massard, et al. J. Clin. Oncol. 2016; 34(26):3119-3125.
National Cancer Institute Common Terminology Criteria for Adverse Events v5.0 (NCI CTCAE) published Nov. 27, 2017 by the National Cancer Institute
National Comprehensive Cancer Network. NCCN clinical practice guidelines in oncology: non-small cell lung cancer. Version 3.2022, available at https://www.nccn.org/.
Oken, et al. Am J Clin Oncol. 1982; 5(6):649-655.
Ostrem, et al. Nature. 2013; 503:548-551.
Ostrem and Shokat. Nature Rev Drug Discov. 2016; 15(11):771-785.
Patricelli, et al. Cancer Discovery. 2016; 6:316-329.
Paz-Ares, et al. N. Engl. J. Med. 2018; 379:2040-2051.
PD-L1 IHC 22C3 pharmDx, Dako North America, Inc. 6392 Via Real, Carpinteria, California 93013, USA, available at https://www.accessdata.fda.gov/cdrh_docs/pdf15/p150013c.pdf, Edition 09/15, last accessed Mar. 31, 2023.
Reck, et al. J. Clin. Oncol. 2019; 37(7):537-546.
Ribas, et al. N. Eng. J. Med., 2013; 368(14):1365-1366
Rini, et al. N. Engl. J. Med., 2019; 380:1116-1127.
Rodriguez-Abreu et al. Ann. Oncol. 2021 July; 32(7):881-895.
Roila, et al. Ann. Oncol. 2016; 27(5):v119v133
Sezer, et al. Lancet 2021; 397(10274):592604.
Schwartz, et al. Eur. J. Cancer. 2016; 62:132-137.
Schwartz, et al. Eur. J. Cancer. 2009; 45:261-267.
Surveillance, Epidemiology, and End Results (SEER) Program (www.seer.cancer.gov) SEER*Stat Database: Incidence—SEER Research Data, 17 Registries, November 2021 Sub (2000-2019)—Linked To County Attributes—Time Dependent (1990-2019) Income/Rurality, 1969-2020 Counties, National Cancer Institute, DCCPS, Surveillance Research Program, released April 2022, based on the November 2021 submission. Kaplan-Meier, Ederer II Method. Last accessed May 5, 2022.
Simanshu, et al. Cell. 2017; 170:17-33.
Therasse, et al. J. Natl. Cancer Inst. 2000; 92(3):205-16.
VENTANA PD-L1 (SP263) Assay, Instructions 2021-10-10, 1020514US Rev 1, Ventana Medical Systems, Inc. 1910 E. Innovation Park Drive Tucson, Arizona 85755 USA, available at https://www.accessdata.fda.gov/cdrh_docs/pdf16/P160046S010C.pdf, last accessed Mar. 31, 2023.
Wen, et al. J. Clin. Oncol. 2010; 28:1963-1972.
World Health Organization. 2020 Statistics. https://www.who.int/news-room/fact-sheets/detail/cancer. Last accessed March 2022.
Yang, et al. J. Thorac. Oncol. 2019; 14(3):553-559.

We claim:

1. A method of treating cancer comprising a KRAS G12C mutation in a human subject in need thereof, the method comprising: administering to the subject (a) a first regimen comprising (i) a therapeutically effective amount of sotorasib, (ii) a therapeutically effective amount of carboplatin and (iii) a therapeutically effective amount of pemetrexed and then administering (b) a second regimen comprising (i) the therapeutically effective amount of sotorasib and (ii) the therapeutically effective amount of pemetrexed.

2. The method of claim 1, wherein the first regimen is administered for a period of 4 cycles.

3. The method of claim 2, wherein each cycle is a period of 21 days.

4. The method of claim 1, wherein the therapeutically effective amount of sotorasib is 240 mg.

5. The method of claim 1, wherein the therapeutically effective amount of sotorasib is 960 mg.

6. The method of claim 5, wherein the sotorasib is administered orally.

7. The method of claim 6, wherein the sotorasib is administered once daily.

8. The method of claim 1, wherein the sotorasib is administered as a solid dosage form.

9. The method of claim 8, wherein the solid dosage form is a tablet.

10. The method of claim 9, wherein the sotorasib is administered as one tablet comprising 240 mg of sotorasib, two tablets each comprising 120 mg of sotorasib, three tablets each comprising 320 mg of sotorasib, four tablets each comprising 240 mg of sotorasib, or eight tablets each comprising 120 mg of sotorasib.

11. The method of claim 1, wherein the therapeutically effective amount of carboplatin is an amount corresponding to 5 mg/(mL×min) (AUC 5) multiplied with the subject's glomerular filtration rate (GFR) in mL/min increased by 25 mL/min.

12. The method of claim 11, wherein the therapeutically effective amount of carboplatin is 750 mg or less.

13. The method of claim 3, wherein the carboplatin is administered intravenously.

14. The method of claim 13, wherein the carboplatin is administered on day 1 of each cycle.

15. The method of claim 1, wherein the subject is not administered carboplatin during the second regimen.

16. The method of claim 1, wherein the therapeutically effective amount of pemetrexed is 500 mg/m².

17. The method of claim 1, wherein pemetrexed is administered intravenously.

18. The method of claim 1, wherein the administration of pemetrexed starts on day 1 of the first regimen.

19. The method of claim 1, wherein the pemetrexed is administered in 21 day intervals during the first regimen and the second regimen.

20. The method of claim 1, further comprising administering to the subject a therapeutically effective amount of folic acid.

21.-24. (canceled)

25. The method of claim 1, further comprising administering to the subject a therapeutically effective amount of vitamin B12.

26.-29. (canceled)

30. The method of claim 1, further comprising administering to the subject a therapeutically effective amount of dexamethasone.

31. (canceled)

32. (canceled)

33. The method of claim 1, wherein the subject is an adult.

34. The method of claim 1, wherein the subject, prior to treatment, has not received a prior systemic anticancer therapy for the cancer (first line treatment).

35. The method of claim 1, wherein the subject has a creatinine clearance (CrCl) of equal or greater than 45 mL/min as calculated by the Cockcroft-Gault formula.

36. The method of claim 1, wherein the subject has an ECOG Performance Status of 0 or 1.

37. The method of claim 1, wherein the cancer exhibits a TC score of less than 1%.

38. (canceled)

39. (canceled)

40. The method of claim 1, wherein the cancer exhibits a PD-L1 tumor proportion score (TPS) of less than 1%.

41. (canceled)

42. (canceled)

43. The method of claim 1, wherein the cancer does not comprise an EGFR or ALK alteration.

44. The method of claim 1, wherein the cancer is lung cancer.

45. The method of claim 1, wherein the cancer is non-small cell lung cancer.

46. The method of claim 1, wherein the cancer is nonsquamous non-small cell lung cancer.

47. The method of claim 46, wherein the nonsquamous non-small cell lung cancer is stage IV or advanced stage IIIB/C.

48.-50. (canceled)
